SEQUENCE LISTING (5 sequences)

SEQ ID NO: 1 - skipped

SEQ ID NO: 2 - DNA, 19 bp, synthetic construct
  source 1..19: mol_type = other DNA
ttttttttttttttttttt

SEQ ID NO: 3-5 - skipped (3 entries)

(19) United States
(12) Patent Application Publication
(10) Pub. No.: US 2025/0313590 A1
(43) Pub. Date: Oct. 9, 2025

(54) Phosphoramidite morpholino monomers towards synthesis/convergent synthesis of PMO, TMO, their chimera oligos in 5 prime to 3 prime direction

(71) Applicant: INDIAN ASSOCIATION FOR THE CULTIVATION OF SCIENCE, Kolkata (IN)

(72) Inventors: Surajit Sinha, Kolkata (IN); Atanu Ghosh, Kolkata (IN); Arpan Banerjee, Kolkata (IN)

(21) Appl. No.: 19/079,082

(22) Filed: Mar. 13, 2025

(30) Foreign Application Priority Data
Mar. 16, 2024 (IN) 202431019488

(51) Int. Cl. C07H 21/00

(52) U.S. Cl. CPC C07H 21/00

(57) ABSTRACT

The present invention relates to 5′-morpholino amidite monomers as 5′-CE/5′-^{t}Bu phosphoramidite morpholino monomers (2) and process chemistry for the efficient synthesis of N-Trityl or monomethoxytrityl (MMTr)-protected 5′-morpholino amidite monomers and their use in the synthesis of phosphorodiamidate morpholino oligonucleotides (PMO), thiophosphoramidate morpholino oligonucleotides (TMO) and their chimeras which can be used for antisense technology or in general oligonucleotides related research.

### CROSS REFERENCE TO RELATED APPLICATION(S)

This application is related to and claims priority to Indian Patent Application No. 202431019488 filed on Mar. 16, 2024, the contents of which are incorporated by reference herein.

### INCORPORATION BY REFERENCE

This application includes a sequence listing in computer readable form (a “XML” file) that is submitted herewith on an XML text file named IASC seq. list file.xml, created on Jun. 30, 2025 and 4,096 bytes in size. This sequence listing is incorporated by reference herein.

The sequence listing presented in XML text file and presented in the attached Applicants response of 30 Jun. 2025 is a part of para [69] presented below and not any new matter, with the opening portion of the sequences stated as PMO/TMO/PMO-TMO/GMO-PMO-TMO indicating the oligonucleotide/oligomer type that are either phosphorodiamidate morpholino oligonucleotides (PMO), thiophosphoramidate morpholino oligonucleotides (TMO) Guanidinium linked Morpholino Oligonucleotides (GMO) or their mix, with the monomers of the oligomers/oligonucleotides linked by g=guanidinium linkage; s=Thiophosphoramidate linkage as shown in the suffix of the sequences (i)-(v) below, said sequences are the sequences of nucleobase thymine (T) attached to the monomer unit forming the oligomer/oligonucleotide.

i.
PMO 5′-TTTTTTT-MMTr 3′
ii.
PMO 5′-TTTTTTTTTTTTTTTTTTT-MMTr 3′
iii.
TMO 5′-TₛTₛT_{Ss}T-Tr (CPh3)-3′
iv.
PMO-TMO chimera 5′-TTTₛTₛT-CPh₃-3′[S stands-TMO linkages]
v.
GMO-PMO-TMO chimera 5′-TgTTₛT-CPh3-3′:

[g = guanidinium linkage; s = Thiophosphoramidate linkage]

### FIELD OF INVENTION

The present invention provides for 5′-morpholino phosphoramidite monomers and process chemistry for the efficient synthesis of N-Trityl or monomethoxytrityl (MMTr)-protected 5′-morpholino phosphoramidite monomers and their industrial utility in the synthesis of phosphorodiamidate morpholino oligonucleotides (PMO), thiophosphoramidatemorpholino oligonucleotides (TMO) and their chimeras which can be utilized for oligonucleotides and antisense technology/antisensetherapeutics based thereon.

### BACKGROUND ART

PMOs are routinely used for selective gene regulation due to their target specificity and very good pharmacokinetics owing to their high endonuclease stability. In 2016, Eteplirsen, the first PMO based drug, was approved by FDA after clinical phase trials for the treatment of Duchenne muscular dystrophy (DMD) and was developed by Sarepta Therapeutics, USA. Since then, three more drugs, namely: Golodirsen, Viltolarsen, and Casimersen have been approved as exon skipping therapies for various mutations.

At present, Gene Tools LLC, USA (www.gene-tools.com) is the only supplier of PMOs, with their patented technology (a. Summerton, J.; Weller, D. U.S. Pat. No. 5,185,444, 1993; b. Weller, D. D.; Hassinger J. N. U.S. Patent 2009/0088562A1).

Their method of preparation is chlorophosphoramidate chemistry where trityl protected chlorophosphoramidate monomers (commonly known as “amidate” monomers) are used and during chain elongation for oligomer synthesis, trityl is deblocked by heterocyclic amine-based acidic salt in a trifluoroethanol containing solvent, the composition of which is patented (c. Weller, D. D. et. al. U.S. Patent 2009/0131632A1). For efficient coupling during chain elongation for LiBr was used.

The current inventors are the second group to develop methods for the synthesis of PMO through (i) improving the chlorophosphoramidate chemistry of Gene Tools and (ii) H-Phosphonate chemistry (Sinha, S. et. al. “Synthesis of Morpholino Monomers, Chlorophosphoramidate Monomers and Solid Phase Synthesis of Short Morpholino Oligomers”. Current Protocols in Nucl. Acid Chem. 2015, 4.65.1-4.65.26 (Wiley); and Sinha, S. et. al. “Synthesis of phosphorodiamidate morpholino oligonucleotides by H-phosphonate method”. Tetrahedron Lett. 2015, 56, 4565-4568 and Sinha, S. et. al. MMTr-protected H-phosphonate monomers and MMTr-deprotection by organic acids are the right combination of morpholino oligonucleotides synthesis (Patent Application No. as 201631037420, 2 Nov. 2016).

In 2023, Wada et. al. reported the convergent synthesis of PMO in solution by H-Phosphonate Chemistry. However, the length of the PMO achieved in this report is only 8-mer (Tsurusaki, T.; Sato, K.; Imai, H.; Hirai, K.; Takahashi, D.; Wada, T. Convergent synthesis of phosphorodiamidate morpholino oligonucleotides (PMOs) by the H-phosphonate approach. Sci. Rep. 2023, 13(1), 12576), which is not suitable for any antisense applications. Moreover, the protocol was not extended for solid-phase synthesis of PMOs.

Recently, Marvin H. Caruthers et. al. reported PMO-DNA chimera synthesis using phosphoramidite(amidite) chemistry (Molecules 2023, 28, 5380), where 5′-dimethoxytrityl (DMTr) protected with 3′-N-phosphoramidite morpholino monomers were used for coupling, followed by boronation using BH₃. Acid-mediated cleavage of DMTr group was performed for chain elongation from 3′→5′ direction. Removal of cyanoethyl group, followed by oxidation using I₂/Me₂NH was performed at the end of the synthesis cycle to obtain phosphorodiamidate backbone.

The chronology to the recently developed and known technologies that exist in the field and the limitations of the same are provided hereunder:

### (i) Synthesis of PMO Using Chlorophosphoramidate Chemistry:

Present technology for making PMOs is patented by Gene Tools LLC using chlorophosphoramidate chemistry. It involves the use of trityl-protected chlorophosphoramidate called “amidate morpholino monomers” which are coupled with 3′-morpholino NH on solid support. In this process, coupling time is significantly high (2-3 hr) because of low reactivity of the pentavalent phosphorous [P(V)] of the amidate monomers. The deprotection of trityl group was done using acetic acid in trifluoroethanol (a. Summerton, J.; Weller, D. U.S. Pat. No. 5,185,444, 1993) (Scheme 1).

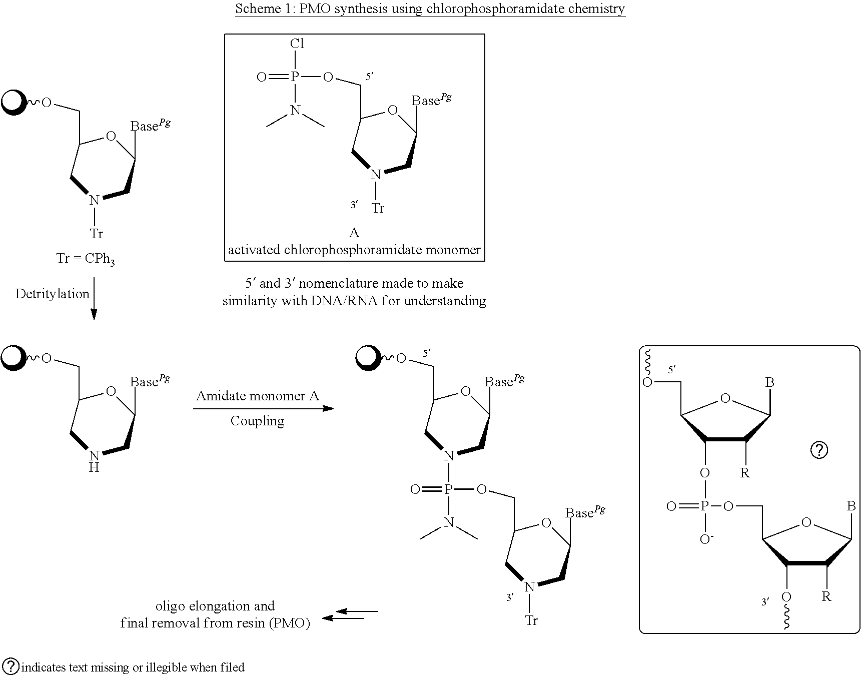

Limitations: Trityl protected chlorophosphoramidate (amidate) monomers are not very stable, particularly in presence of organic or inorganic base in organic solvents and are decomposed within 1 hr. Long-time storage in solid form requires perfectly inert argon atmosphere without which decomposition occurs. LiBr is used as an activator in the coupling step and takes 2-3 hr per coupling. Therefore, it becomes a problem for a longer oligomer synthesis, which is typically required for biological applications. To complete the 25-mer synthesis, 2×25=50 hrs is required only for coupling. Hence, solution of activated monomers has to be prepared freshly for each addition. Hence, the method is not amenable to automated DNA Synthesizer. This demands the development of highly efficient coupling protocol with lower coupling time, in which activated monomers will remain intact in solution. Furthermore, the synthesis is performed using polystyrene resins in DMF and is not compatible with acetonitrile and CPG supports, commonly used for DNA/RNA synthesis.

At present Gene Tools LLC is the only commercial source of PMO that were synthesized as per the synthetic protocol in their patent (a. U.S. Pat. No. 5,185,444, 1993). Hence, an alternative, efficient synthetic protocol is necessary for the synthesis of PMO.

(ii) Improved the Above Method for the Synthesis of PMO Using Chlorophosphoramidate (Amidate) Chemistry from the Present Inventors:

The synthesis of PMO was recently reported using chlorophosphoramidate chemistry using both trityl and Fmoc protecting groups and coupling efficiency was improved in the presence of 5-ethylthio-1H-tetrazole (ETT) as an activator (Scheme 2). This protocol is well-compatible with automated DNA synthesizer. [J. Kundu, A. Ghosh, U. Ghosh, A. Das, D. Nagar, S. Pattanayak, A. Ghose and S. Sinha: Synthesis of Phosphorodiamidate Morpholino Oligonucleotides Using Trityl and Fmoc Chemistry in an Automated Oligo Synthesizer. J. Org. Chem. 2022, 87, 15, 9466-9478 and Synthesis of Fmoc protected morpholino monomers and their use in the synthesis of morpholino oligomer. Application no. 201931044056, 31 Oct. 2019, Filed for US patent 30 Oct. 2020, US20210130379A1].

Limitations: The synthesis is again carried out by polystyrene resins and DMF or NMP solvents. In addition, 5′-chlorophosphoramidate activation of the morpholino oligomer fragments or block activation such as dimer, trimer block etc is not possible, limiting its application for the convergent synthesis of PMO.

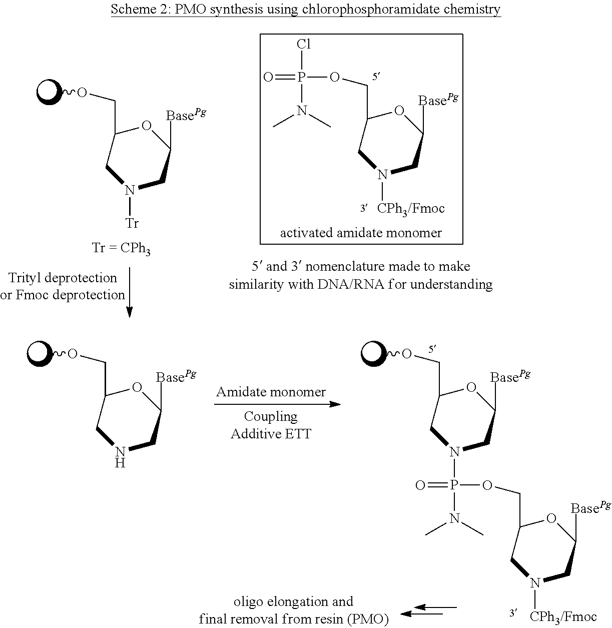

(iii) Synthesis of PMO Using Phosphoramidite (Amidite) Chemistry (Reported from Marvin H. Caruthers Group):

Phosphoramidite chemistry is routinely used for the synthesis of DNA/RNA in an automated DNA synthesizer. Marvin Caruthers et al. applied this chemistry for PMO synthesis. In DNA/RNA synthesis, 3′-terminal of sugar unit is attached with solid supports and 5′-OH is protected with DMTr (Dimethoxytrityl). 5′-DMTr is deprotected and coupled with 3′-phosphoramidite monomer (DNA amidites Y, FIG. 1: Morpholino amidites used for PMO synthesis by Caruthers et. al. DNA amidites used for DNA synthesis) in the presence of tetrazole and followed by oxidation to get dimer. Tetrazole first protonated the diisopropylamine which is then released from “P” center and this electrophilic “P” center is attacked by 5′-OH of solid-support bound nucleoside. This is the procedure is followed for long chain DNA/RNA synthesis (Scheme 3).

Following this chemistry, Marvin H. Caruthers made 3′-N-morpholino phosphoramidite (X, FIG. 1) (amidite monomers) which is then coupled with 5′-OH of support bound ribonucleosides to get the dimer where first unit was deoxyribosugar moiety. In this case BH₃ was used as boronating agent to get the boronate oligos, followed by removal of cyanoethyl group (1:1 TEA:ACN) and final oxidation by I₂/Me₂NH at the end of the synthesis cycle to give DNA-PMO chimera because first unit in solid support was DNA unit (Scheme 4).

Limitations: In this method, all the nucleosides were protected with BIBS group, unlike canonical amide protecting groups in regular DNA/RNA monomers. Hence synthesis is not straightforward and costly. In the case of tetrazole-mediated activation, protonation is shown in di-isopropylamine in the “P” center like DNA/RNA monomers (Scheme 4, Path A). However, there is a chance of protonation of the morpholino N instead of diisopropylamine component, which leads to truncated product and is a major drawback of this method (Scheme 4, Path B). Moreover, the method involves an extra boronation step for chain elongation, leading to major deviation from regular DNA/RNA synthesis. This includes the incompatibility of the method with regular CPG support and canonical amide-based nucleobase protections (such as benzamide for A and C; iso-butyramide for G), used for DNA/RNA synthesis. Additionally, they had to modify the deblocking conditions (0.5% TFA/10% TMPB in CHCl₃). Finally, for the deprotection of BIBS groups, they had to use a fluoride-based reagent, which compelled them to modify their support of choice from CPG to polystyrene resins. So, except for using the amidite chemistry, most of the steps are different from the conventional DNA/RNA synthesis protocol. Also, the oligo-synthesis commenced with a DNA monomer loaded solid support, which always resulted in the morpholino oligos with a first unit being a DNA monomer. Hence, the ultimate product is DNA-PMO hybrid (Scheme 4).

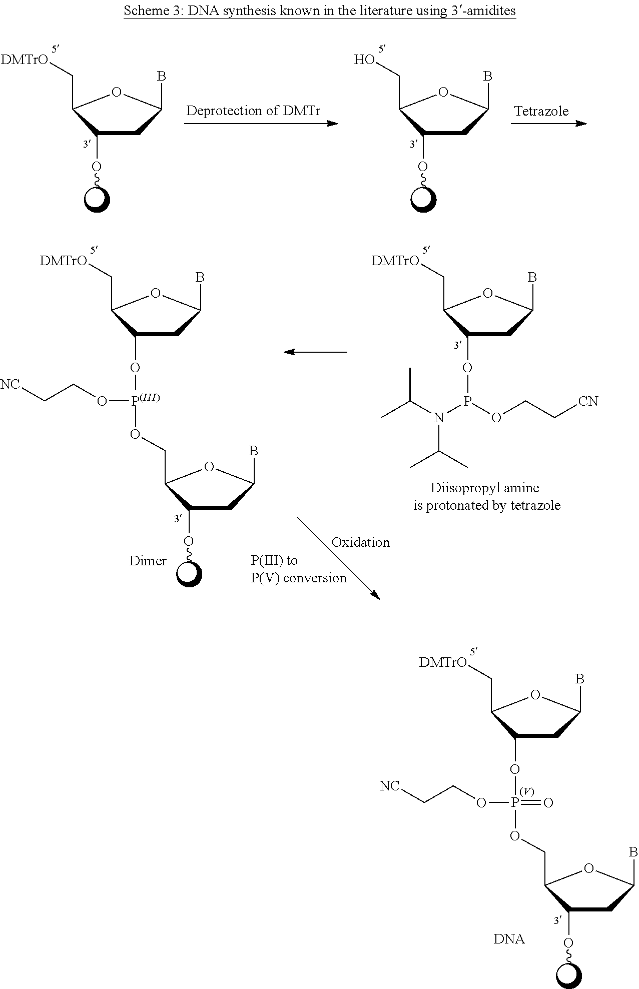

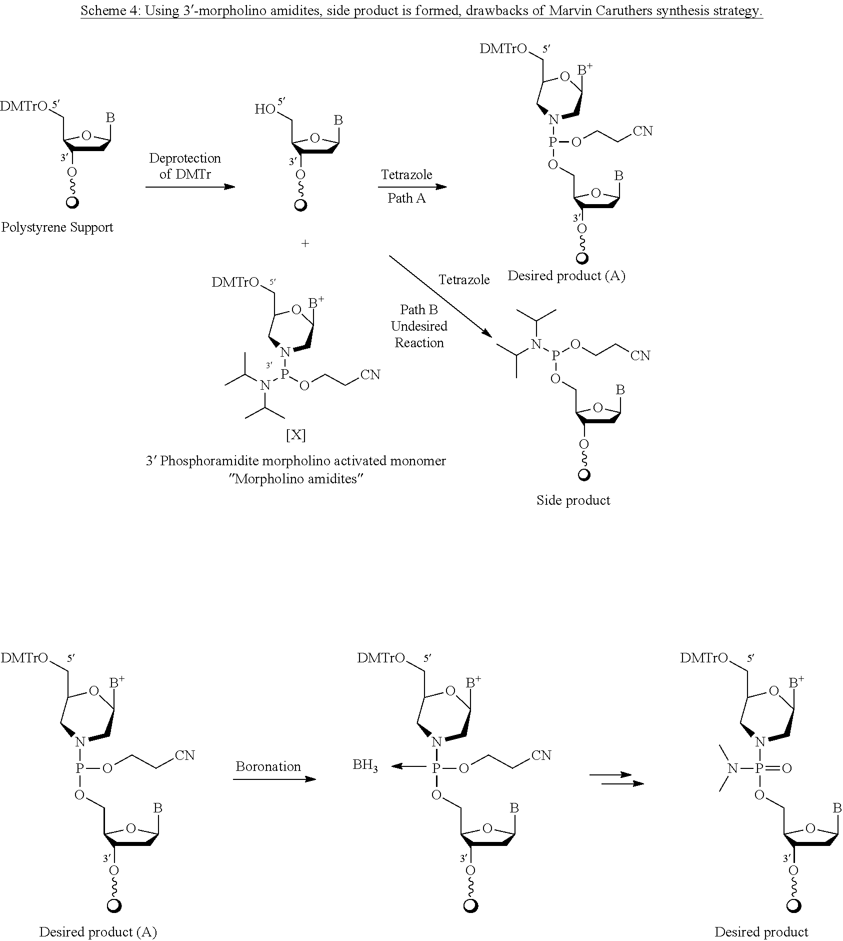

Thus, unlike Caruthers method projected above, there is a need to explore on amidite chemistry for PMO synthesis in a relatively simpler approach, to avoid Caruthers' method where per-step boronation of the P(III) state was involved as an extra step. Moreover, Caruthers' method required the use of bis(tert-butyl)isobutylsilyl(BIBS) group for nucleobase protection in the synthesis of PMO, hence providing for amidites that cannot be made commercially available as it is not compatible with standard amide protecting groups, commonly used in the case of DNA/RNA synthesis, and hence they could not be easily deprotected under standard ammonia deprotection conditions requiring the involvement of extra steps and reagents. Further to the above there was a reason to explore for 5′-morpholino amidite monomers that are advantageous over 3-morpholino amidite monomers, as there is no chance of having truncated PMO during the synthesis with 5′-morpholino amidite monomers, since the tetrazole activator can selectively activate the N,N-diisopropyl component of it. In addition there was a need to explore Controlled Pore Glass (CPG), commonly used for DNA/RNA synthesis, as the support of choice for PMO synthesis as the previous method was CPG incompatible and polystyrene support had to be used for PMO synthesis.

Further the problem in the art that was imperative to tackle is that in said Caruthers' report, only PMO-DNA chimera could be obtained whereas regular PMO backbone is the requirement in the art. So exploring monomers and the method for synthesizing the PMO based on such monomers can be useful, that would provide for the regular PMO backbone, which will also be useful for the synthesis of Thiophosphoramidate Morpholino Oligonucleotides (TMOs) and various biologically relevant chimeric MO backbones, such as PMO-TMO, GMO-TMO, GMO-PMO-TMO, (Guanidinium linked Morpholino Oligomer, GMO) which were inaccessible otherwise. So, such monomers is required to be explored that would not only be limited to the synthesis of these chimeras but also would allow coupling with DNA/RNA amidites to get chimeras with DNA/RNA backbone. There is also a need to provide for monomers and method of synthesis thereof that would provide the opportunity of activating PMO blocks or called block activation for the synthesis of full-length PMOs in convergent approach. This convergent approach minimizes the coupling steps on solid support, thus effectively increase PMO synthesis yield with minimal consumption of costly monomers to be useful for scale-up of PMO synthesis. Such block activation could be used for the convergent synthesis of PMO in solution phase also.

Key words: Tr/MMTr-chemistry (Trityl, monomethoxy trityl), morpholino monomers, morpholino phosphoramidite monomers, morpholino oligomers, Morpholino chimeras, solid phase method, antisense reagents.

### OBJECTS OF THE INVENTION

It is thus an object of the present invention to provide for 5′-morpholino amidite monomers and process chemistry for the efficient synthesis of N-Trityl or monomethoxytrityl (MMTr)-protected 5′-morpholino amidite monomers so that they remain industrially utilizable towards the synthesis of oligonucleotides including phosphorodiamidate morpholino oligonucleotides (PMO), TMOs and their chimeras useful for antisense technology.

It is another object of the present invention to provide for monomers for the synthesis of PMO that would roll in 5′→3′ direction, utilizing stepwise oxidation with I₂/Me₂NH to directly access the phosphorodiamidate backbone.

It is yet another object of the present invention to provide for said monomers that would not only be limited towards the synthesis of PMO, TMO type chimeras but would also facilitate coupling with DNA/RNA amidites to get chimeras with DNA/RNA backbone.

It is still another object of the present invention to provide for said monomers leading to DNA/RNA amidites having 3′-DMTr protected 5′-amidites that would be stable and commercially available.

It is yet another object of the present invention to provide for said monomers and process chemistry thereof towards its synthesis and towards the synthesis of phosphorodiamidate morpholino oligonucleotides (PMO) therefrom that would be compatible with standard amide protecting groups, commonly used in the case of DNA/RNA synthesis, to be easily deprotected under standard ammonia deprotection conditions, where no extra steps and reagents would be required to be involved.

It is still another object of the present invention to provide for said monomers for PMO synthesis that would involve controlled Pore Glass (CPG), commonly used for DNA/RNA synthesis, as the choice of support for PMO synthesis over polystyrene support.

It is yet another object of the present invention to provide for monomers that would provide for regular PMO backbone, unlike Caruthers' where only DNA-PMO chimera could be obtained.

It is another object of the present invention to provide for said monomers and TMO, PMO-TMO or several other chimeras based thereon that would allow tuning the oxidation steps where P—NMe₂, P—S, P—O, P═N bonds can be formed.

It is yet another object of the present invention to provide for said monomers facilitating such oligos and chimeras based on convergent approach of synthesis through fragment or block activation by phosphoramidite chemistry.

### SUMMARY OF THE INVENTION

Thus according to the basic aspect of the present invention there is provided said monomers and phosphorodiamidate morpholino oligonucleotides (PMO) thereof comprising 5′-phosphoramidite morpholino monomers (2) as Trityl or monomethoxytrityl (MMTr)-protected 5′-phosphoramidite morpholino monomers as represented hereunder:

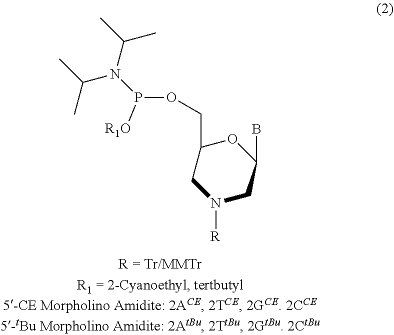

Preferably in said monomers said nucleobase (B) in said monomers includes all four nucleobases: adenine, thyamine, gunanine, cytosine based monomers for each 2-cyanoethyl (CE) and tert-butyl (^{t}Bu) based phosphoramidites.

Advantageously said monomers are stable in anhydrous acetonitrile solvent up to 3 days in room temperature.

According to another aspect of the present invention there is provided a process for the synthesis of monomers and phosphorodiamidate morpholino oligonucleotides (PMO) thereof comprising the steps of
(i) Providing 3′-NH, 5′-OH morpholino monomers with nucleobases protected with regular amide-based protecting groups including benzamide for A and C, iso-butyramide for G;
(ii) Protecting the 3′-NH end with Trityl or monomethoxytrityl (MMTr) groups to obtain 3′-N Trityl or monomethoxytrityl (MMTr) protected 5′-OH morpholino monomers;
(iii) Adding 2-cyanoethyl-N,N,N′,N′-tetraisopropylphosphorodiamidite reagent (for 5′-CE Morpholino Amidites) or tert-butyl-N,N,N′,N′-tetraisopropylphosphorodiamidite reagent (for 5′-^{t}Bu Morpholino Amidites) to said 3′-N Trityl or monomethoxytrityl (MMTr) protected 5′-OH morpholino monomers dissolved in THF-ACN (8:2) or THF:DCM (8:2) or DCM in presence of activators to activate N,N-diisopropyl component of said tetraisopropylphosphorodiamidite reagent at room temperature and obtaining therefrom 5′-CE/5′-^{t}Bu based said 5′-phosphoramidite morpholino monomers (2) by quenching the reaction mixture by saturated NaHCO₃ and extracting with solvent including ethyl acetate to further enable dimers and phosphorodiamidate morpholino oligonucleotides (PMO) therefrom.

Preferably in said process for the synthesis of monomers and phosphorodiamidate morpholino oligonucleotides (PMO) thereof wherein said activators are 5-Ethylthio-1H-tetrazole (ETT) or 5-Benzylthio-1H-tetrazole (BTT) as activator, in combination with N-methyl imidazole (NMI).

According to another preferred aspect of the process for the synthesis of monomers and phosphorodiamidate morpholino oligonucleotides (PMO) thereof wherein for preparation of said dimers by chain extension at 3′-NH end of morpholino said 5′-CE/5′-^{t}Bu phosphoramidite morpholino monomers (2), and, 5′-TBDPS (tert-butyldiphenylsilyl) or DMTr (Dimethoxytrityl) protected morpholino 3′-NH monomers are taken in MeCN solution in presence of said ETT or BTT and in combination with said N-methyl imidazole (NMI) as activators of said N,N-diisopropyl component of said 5′-CE/5′-^{t}Bu Morpholino amidite monomers (2) for forming the P—N bond in about ˜10 mins, followed by oxidation of the P(III) centre to P(V) centre by employing I₂ and dimethyl amine (Me₂NH)≈2.0M in THF as oxidizing agent to produce the desired phosphorodiamidate dimer (4) that is Trityl or monomethoxytrityl (MMTr) dimer.

Preferably in said process for the synthesis of monomers and phosphorodiamidate morpholino oligonucleotides (PMO) thereof said phosphorodiamidate morpholino oligonucleotides (PMO) is synthesized in automated Oligo synthesizer for solid phase synthesis by employing 5′-Morpholino Amidites (2) as Trityl or monomethoxytrityl (MMTr)-protected 5′-phosphoramidite morpholino monomers (2) based on the steps of:

attaching the 5′-OH morpholino monomers to (Controlled pore glass)/polystyrene support having nucleobases protected with regular amide-based protecting groups including benzamide for A and C, iso-butyramide for G to CPG and having morpholino 3′-NH end protected with Trityl or monomethoxytrityl (MMTr) to allow chain extension at said 3′-NH end of morpholino;
deblocking the Trityl protection with 2% CYPMSA (3-Cyanopyridine-Methanesulfonic Acid salt) or monomethoxytrityl (MMTr) protection with 3% TCA (trichloroacetic acid) in DCM;
coupling with 5′-^{t}Bu-phosphoramidite morpholino monomers (2) of about 0.2 M concentration as Trityl or monomethoxytrityl (MMTr)-protected 5′-^{t}Bu-phosphoramidite morpholino monomers in presence of ETT, NMI, followed by oxidation of P(III) to P(V) state in presence of 0.05M I₂/2M Me₂NH in THF to obtain the dimer, followed by, capping N-end of the dimer away from the support by N,N-Diisopropylethylamine/acetic anhydride and continuing the cycle to obtain said phosphorodiamidate morpholino oligonucleotides (PMO) therefrom having —P—NMe₂ linkages at P(V);
cleaving the thus obtained phosphorodiamidate morpholino oligonucleotides (PMO) from the solid support by 30% aqueous NH₃ at 55° C. for 16 h.

Preferably in said process for the synthesis of monomers and phosphorodiamidate morpholino oligonucleotides (PMO) thereof said phosphorodiamidate morpholino oligonucleotides (PMO) as Thiophosphoramidate Morpholino Oligonucleotides (TMO) are synthesized by involving 5′-CE morpholino amidites as Trityl or monomethoxytrityl (MMTr)-protected 5′-CE-phosphoramidite morpholino monomers (2) followed by said oxidation of P(III) to P(V) state in presence of 3-[(Dimethylaminomethylene)amino]-3H-1,2,4-dithiazole-5-thione (DDTT) (0.1M) in pyridine, and obtaining therefrom said dimers and Thiophosphoramidate Morpholino Oligonucleotides (TMO) thereof;
said phosphorodiamidate morpholino oligonucleotides (PMO) as SulfonylphosphoramidateMorpholino Oligonucleotides are synthesized by involving 5′-CE morpholino amidites as Trityl or monomethoxytrityl (MMTr)-protected 5′-phosphoramidite morpholino monomers (2) followed by said oxidation of P(III) to P(V) state in presence of RSO₂N₃/Aryl sulfonyl azide, where R includes Long chain alkyl group, benzyl, methyl, ethyl in MeCN or MeCN-THF, and obtaining therefrom said dimers and SulfonylphosphoramidateMorpholino Oligonucleotides thereof;
said dimers and incorporation of monomers in continuing cycle of liquid/solid phase synthesis enables attainment of oligonucleotide chimers including Guanidinium linked Morpholino Oligomer (GMO) containing chimeras, PMO-TMO chimers, GMO-PMO-TMO chimers, PMO-sulfonylphosphoramidate chimers, phosphoramidate-phosphorodiamidate chimers.

More preferably in said process for the synthesis of monomers and phosphorodiamidate morpholino oligonucleotides (PMO) thereof wherein preparation of dimers and morpholino oligonucleotides (PMO) thereof by chain extension at 5′-OH end of morpholino towards convergent synthesis leading to 3-6 mer PMO fragments in solution or in solid phase is based on the steps of:
keeping free —OH at the 5′ end of morpholino and keeping protected -3′ N end of morpholino by Trityl group which said —OH at 5′ end is then activated to provide 5′-phosphoramidite PMO fragment block that is said trityl protected at its -3′ N end; allowing convergent coupling of said 5′-phosphoramidite PMO fragment block, with, Trityl or monomethoxytrityl (MMTr) de-protected morpholino 3′-NH monomer/oligomer chain with free 3′-NH end; followed by
   oxidation, and repeating the cycle, and obtaining therefrom phosphorodiamidate backbone of 3-6mer PMO fragments (Block) in solution.

According to yet another preferred aspect of the process for the synthesis of monomers and phosphorodiamidate morpholino oligonucleotides (PMO) thereof said Trityl or monomethoxytrityl (MMTr) de-protected morpholino 3′-NH monomer/oligomer chain with free 3′-NH end is attached to solid support at its other end through 5′-O— for said convergent coupling to attain therefrom diverse chimeric backbone based PMOs including thiophosphoramidate, phosphorodiamidate, phosphoramidate and sulfonyl-phosphorodiamidate linkages also including PMO-DNA/PMO-RNA chimera.

Preferably in said process for the synthesis of monomers and phosphorodiamidate morpholino oligonucleotides (PMO) thereof wherein MMTr-protecting group based on mild acid treatment including 2-3% TCA (Trichloroacetic acid) in DCM/3% DCA (Dichloroacetic acid) in DCM, enabled deprotection to generate free —NH for further coupling.

More preferably in said process for the synthesis of monomers and phosphorodiamidate morpholino oligonucleotides (PMO) thereof wherein said oxidizing agents for Thiophosphoramidate backbone involves 3-[(Dimethylaminomethylene)amino]-3H-1,2,4-dithiazole-5-thione (DDTT) in pyridine; for Phosphoramidate backbone involves (1S)-(+)-(10-camphorsulfonyl)-oxaziridine (CSO) in anhydrous MeCN and for Sulfonylphosphorodiamidate backbone involves said RSO₂N₃ in MeCN or MeCN-THF.

### BRIEF DESCRIPTION OF FIGURES

FIG. 1: Morpholino amidites (X) used for PMO synthesis by Caruthers et. al. DNA amidites (Y) used for DNA synthesis;

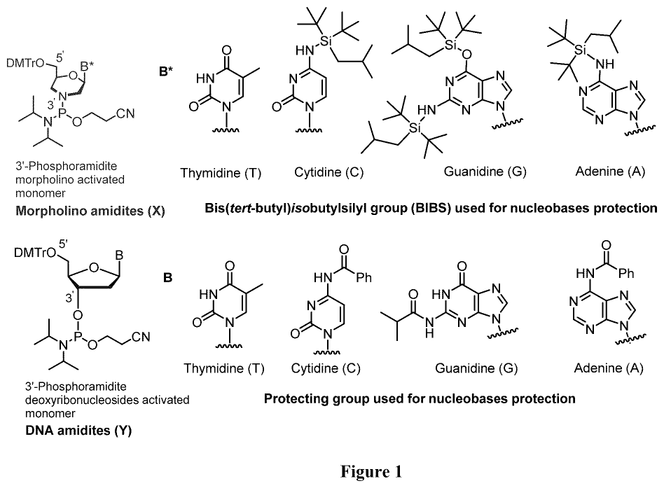

FIG. 2: HRMS (ESI) [M+Na]⁺: Calculated mass for C₅₆H₆₄N₇O₉PSiNa=1060.4170, found 1060.4171;

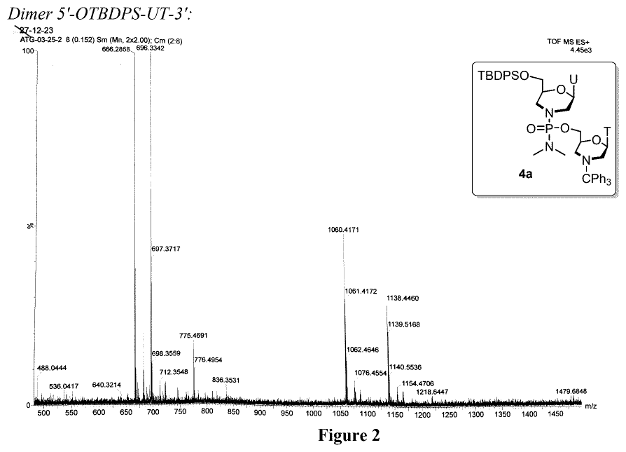

FIG. 3: HRMS (ESI) [M+Na]⁺: Calculated mass for C₆₂H₆₆N₇O₁₁PNa=1138.4456, found 1138.4456;

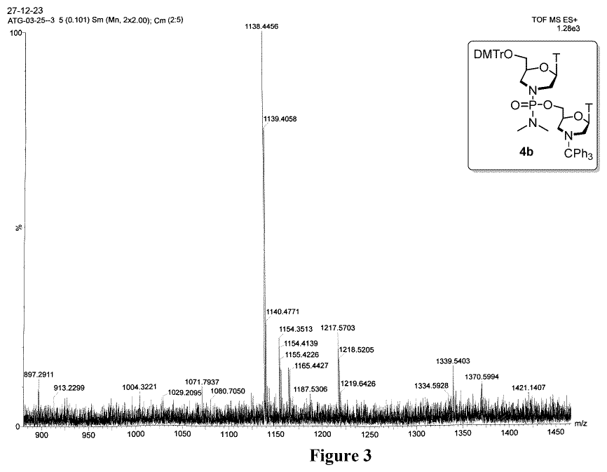

FIG. 4: HRMS (ESI) [M+Na]⁺: Calculated mass for C₃₄H₅₃N₁₁O₁₄P₂Na=924.3146, found 924.3145;

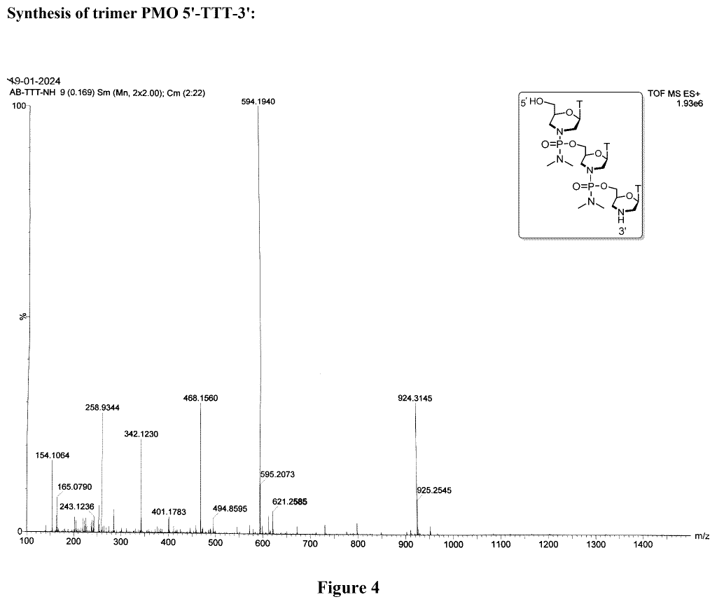

FIG. 5: MALDI-TOF MS [M+2Na]⁺: Calculated mass for C₈₂H₁₂₉N₂₇O₃₄P₆Na₂=2267.742, found 2266.309. (MMTr was deprotected while matrix sample preparation, hence mass was obtained without MMTr group);

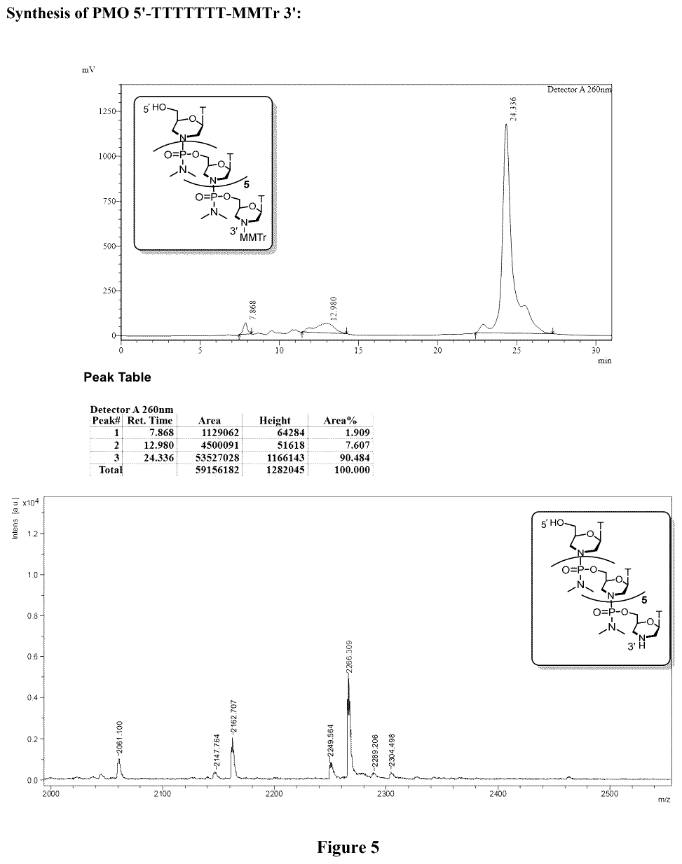

FIG. 6: HRMS (ESI) [M+H]⁺: Calculated mass for C₅₉H₇₁N₁₂O₁₉P₃S₃=1441.3438, found 1441.3411;

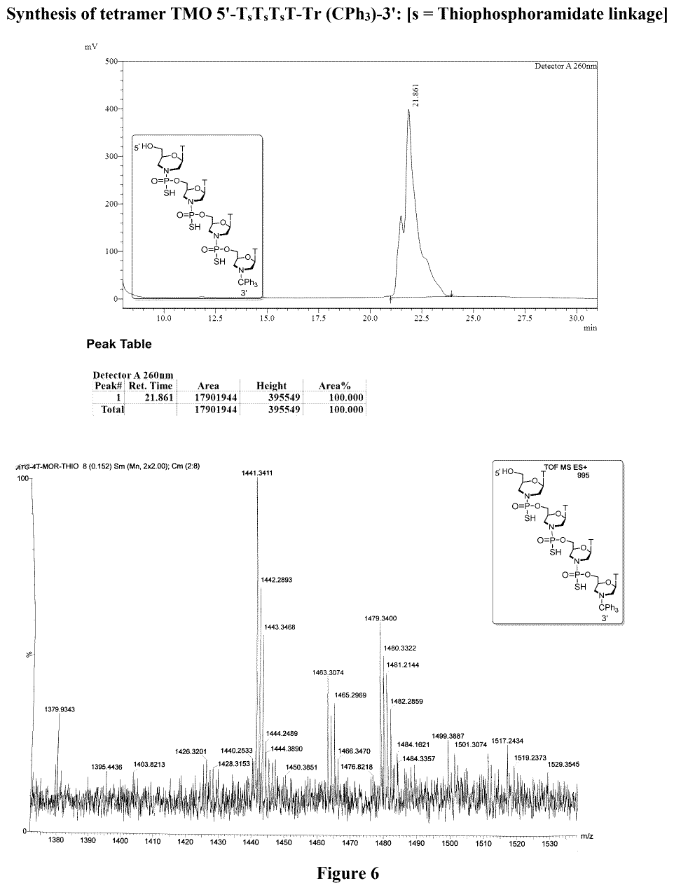

FIG. 7: HRMS (ESI) [M+H]⁺: Calculated mass for C₆₂H₈₄N₁₃O₁₅P₃=1344.5469, found 1344.5500;

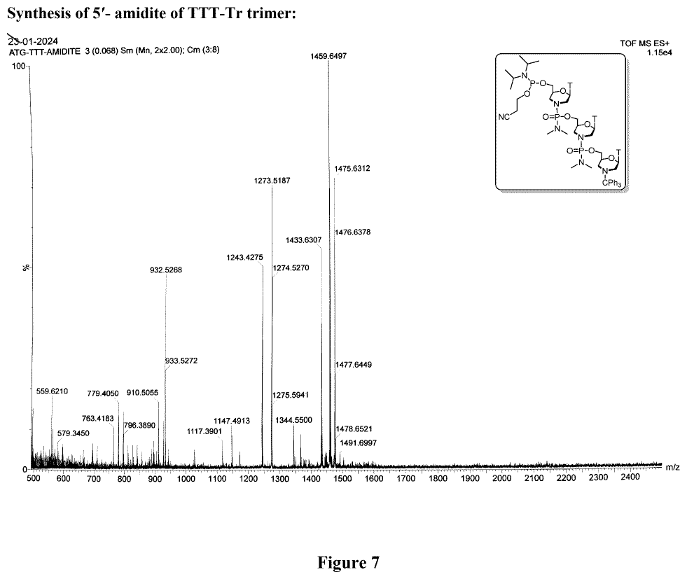

FIG. 8: HRMS (ESI) [M+Na]⁺: Calculated mass for C₈₀H₁₀₂N₁₅O₁₉P₃SiNa=1720.6356, found 1720.6355;

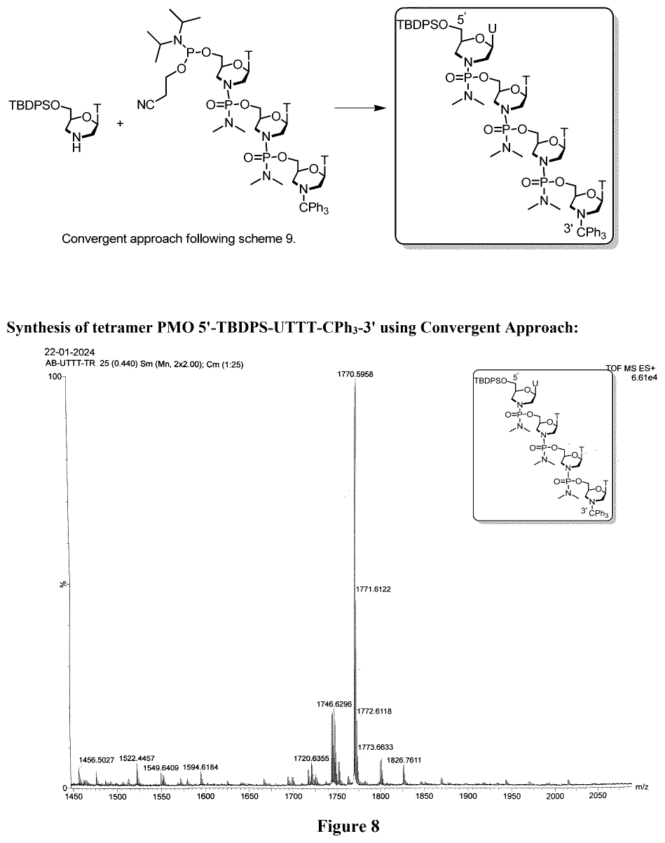

FIG. 9: HRMS (ESI) [M+Na]⁺: Calculated mass for C₇₃H₉₅N₁₇O₂₄P₄S₂Na=1804.5025, found 1804.5192;

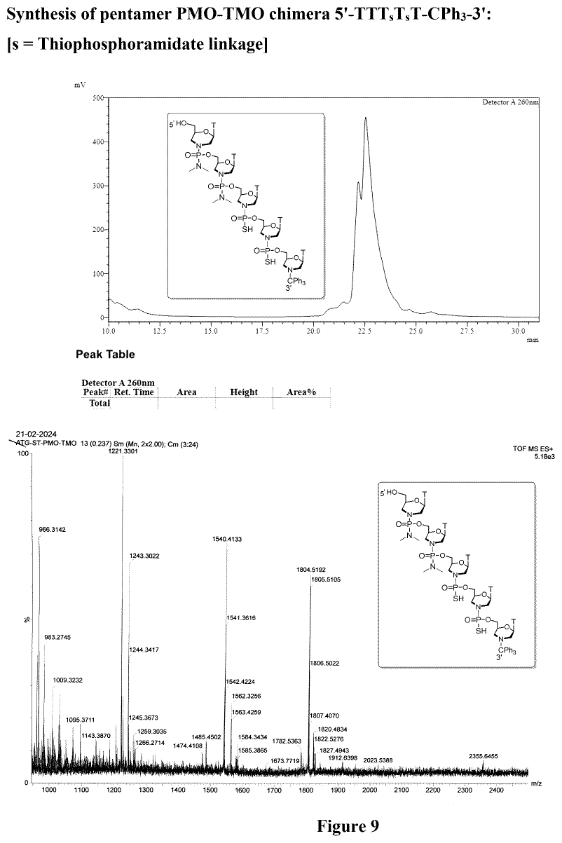

FIG. 10: HRMS (ESI) [M+H]⁺: Calculated mass for C₆₂H₇₇N₁₅O₁₇P₂S=1398.4922, found 1398.4895;

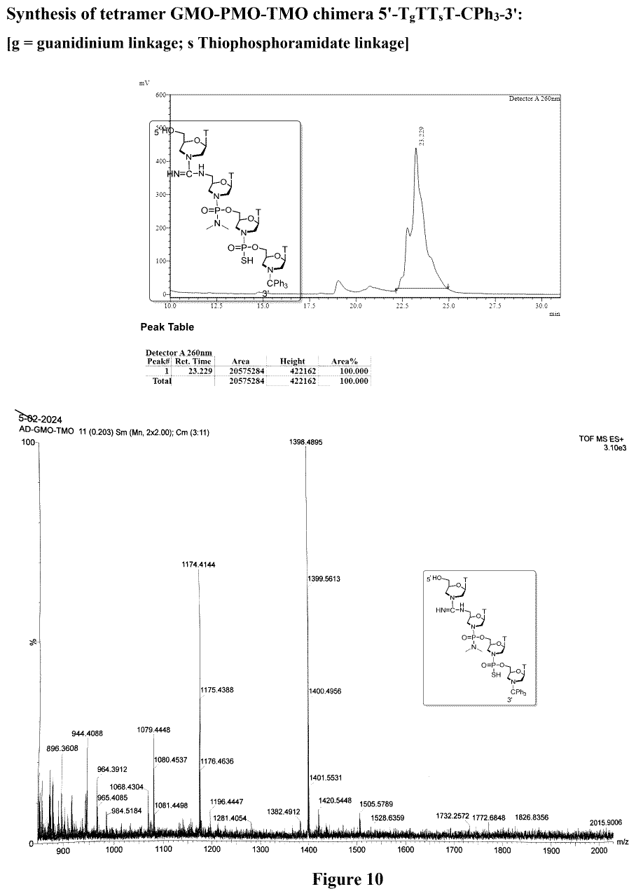

FIG. 11: MALDI-TOF MS [M+2Na]⁺: Calculated mass for C₂₂₆H₃₅₇N₇₅O₉₄P₁₈Na₂=6232.280, found 6231.673. (MMTr was deprotected while matrix sample preparation, hence mass was obtained without MMTr group).

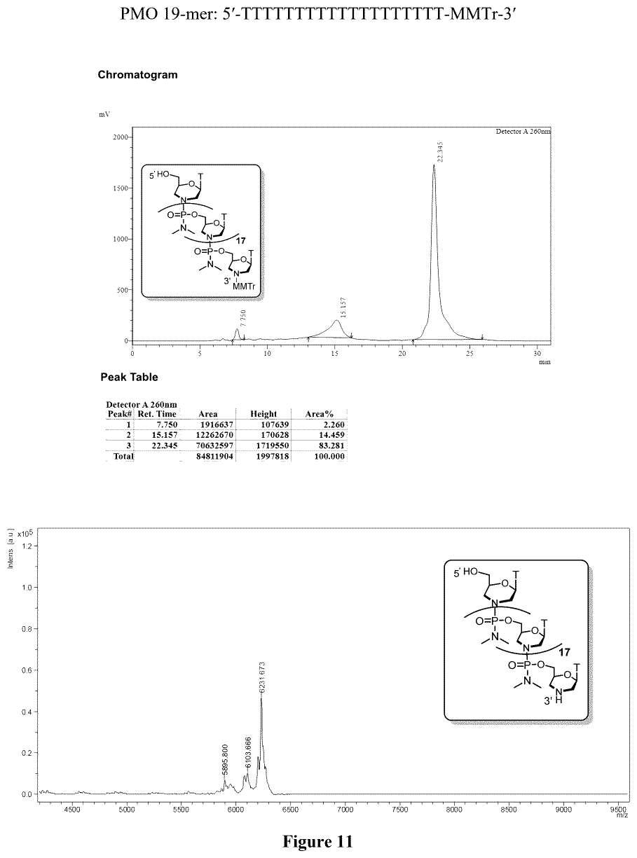

### DETAILED DESCRIPTION OF THE INVENTION

As discussed hereinbefore the present invention provides for 5′-morpholino amidite monomers and process chemistry for the efficient synthesis of N-Trityl or monomethoxytrityl (MMTr)-protected 5′-morpholino amidite monomers and their industrial utility in the synthesis of phosphorodiamidate morpholino oligonucleotides (PMO) and several other chimeras utilized for oligonucleotides and antisense technology/antisensetherapeutics based thereon.

### EXAMPLES

### Synthesis of PMO Using 5′-Morpholino Amidites as Activated Monomers for Chain Elongation

The present invention disclosure provides a relatively simpler method for the synthesis of phosphorodiamidate morpholino oligonucleotides (PMOs) using amidite chemistry. The synthesis is robust and simple and rolls in 5′→3′ direction. It begins with the coupling of 5′-morpholino amidites with morpholino 3′-NH, followed by oxidation using I₂/Me₂NH. 5-Ethylthio-1H-tetrazole (ETT), in combination with N-methyl imidazole (NMI), has been used as the selective activator of the N,N-diisopropyl component of the morpholino amidites during the coupling step and there is no chance of having truncated oligos here. In the present invention 5′-morpholino amidites (2), has been involved unlike Caruthers' 3′-morpholino amidites.

The present protocol comes with the advantage of convenient transfer to automated Oligosynthesizersince the synthesis is being done on CPG support with coupling of 5′-amidite monomers, followed by oxidation with I₂/Me₂NH to directly access the phosphorodiamidate backbone. 5′-morpholino amidites (2) are synthesized from 5′-OH morpholino monomers of all four nucleobases with regular amide-based protecting groups (such as benzamide for A and C; iso-butyramide for G), using 2-cyanoethyl-N,N,N′,N′-tetraisopropylphosphorodiamidite (for 5′-CE Morpholino Amidites)/tert-butyl-N,N,N′,N′-tetraisopropylphosphorodiamidite (for 5′-^{t}Bu Morpholino Amidites) in THF-ACN or THF-DCM mixture or DCM, in the present case THF-ACN (8:2) in presence of 5-Benzylthio-1H-tetrazole (BTT) as an activator was used (Scheme 5).

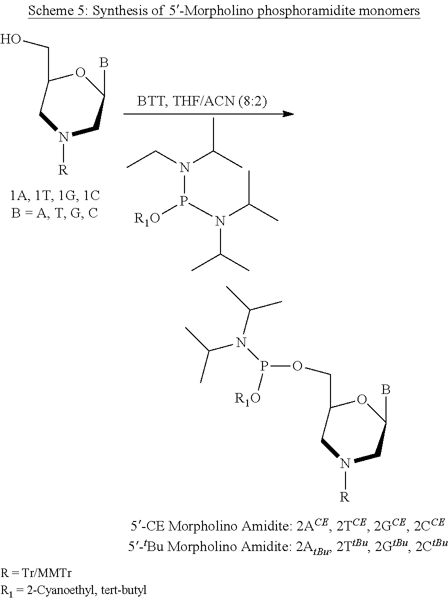

The present protocol is fundamentally different from Caruthers' method as depicted here. In this method, coupling of 5′-morpholino amidites are being done with the morpholino 3′-NH, rolling the synthesis in 5′→3′ direction. Therefore, there is selective protonation of the N,N-diisopropyl unit by ETT/BTT activator, hence, no truncated product is formed, making it a robust and high yielding method for the synthesis of PMO on automated commercial DNA synthesizers. Synthesis is started with morpholino unit, not with DNA unit to get the regular PMO, unlike DNA-PMO chimera in Caruthers' method (Scheme 6). Moreover, CPG has been used as support of choice for the seamless synthesis of PMO on Oligosynthesizer, like regular DNA/RNA.

To standardize our method, we first coupled the 5′-TBDPS or DMTr protected morpholino 3′-NH monomers with 5′-morpholino amidites in MeCN solution, in presence of ETT or BTT as activator, in combination with N-methyl imidazole (NMI). This was followed by oxidation of the P(III) centre using I₂ and dimethyl amine (Me₂NH) to produce the desired phosphorodiamidate dimer (Scheme 6).

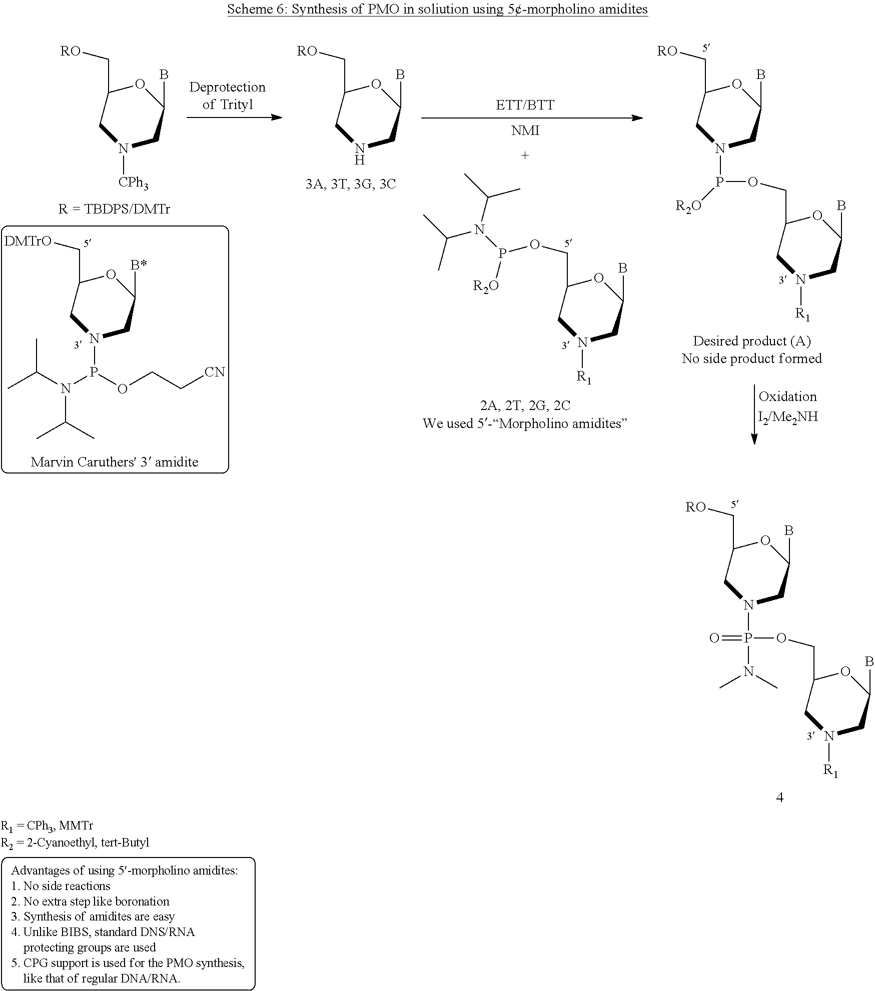

### PMO Synthesis in Automated OligoSynthesizer Using 5′-Morpholino Amidites

After accomplishing the dimer synthesis in solution, we started out to transfer this protocol to automated Oligo Synthesizer to optimize the solid phase synthesis of PMO using both 5′-CE morpholino amidites and 5′-^{t}Bumorpholino amidites. Unfortunately, 5′-CE morpholino amidites failed to give the desired PMO. This is probably due to incomplete removal of the cyanoethyl group under oxidation conditions (I₂/Me₂NH) in solid support, which hindered the oxidation of P(III) to P(V) state, the result of which is reflected as undesired truncated products. Therefore, we proceeded with the 5′-^{t}Bu morpholino amidites to synthesize PMO. It resulted in the desired PMO in excellent yields, under the same oxidation conditions (I₂/Me₂NH). In the present case, 0.05M I₂/2M Me₂NH in THF was used. The PMO synthesis cycle is as outlined in Scheme 7.

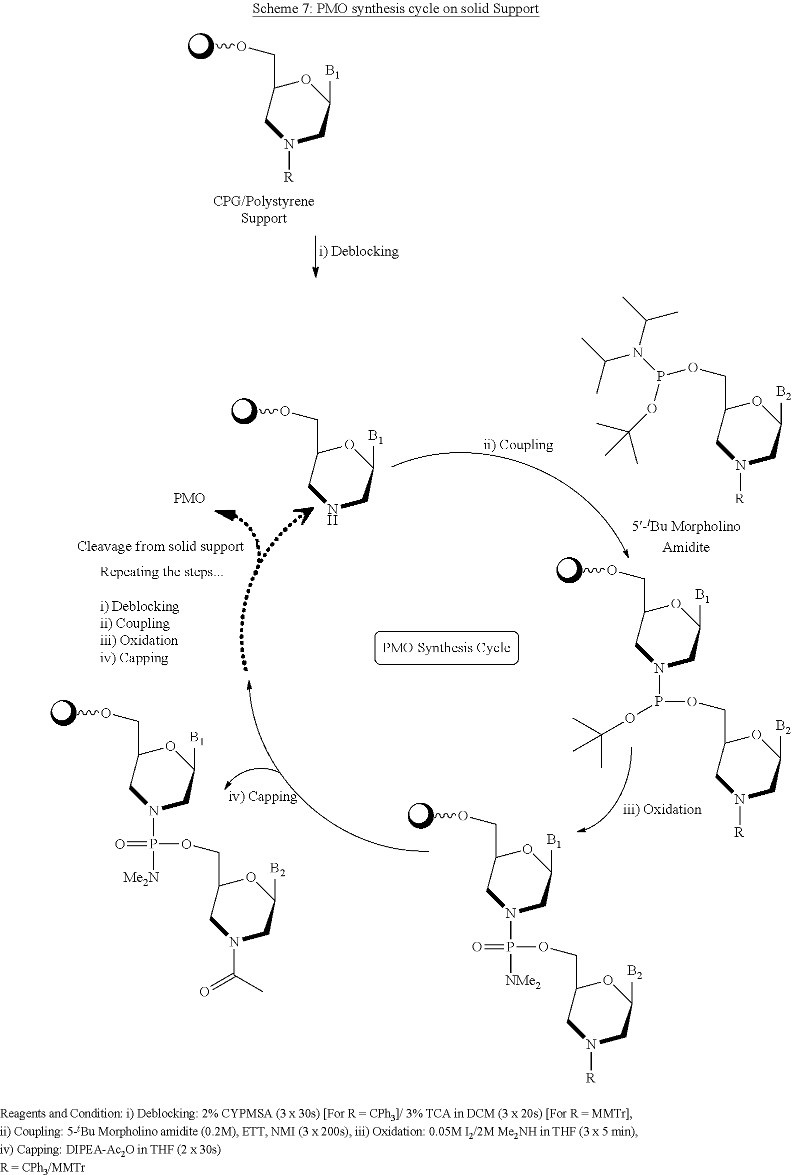

### Synthesis of TMO and Other Chimeric Backbone

The 5′-CE morpholino amidites can also be used to synthesize other different morpholino backbone using diverse oxidising agents. To demonstrate the diverse application of this method, ThiophosphoramidateMorpholino Oligonucleotides (TMO) were synthesized using this 5′-CE morpholino amidites followed by oxidation using 3-[(Dimethylaminomethylene)amino]-3H-1,2,4-dithiazole-5-thione (DDTT) (0.1M) in pyridine. This approach can also be extended to synthesize various biologically important morpholino based chimeric backbones, such as the Guanidinium linked Morpholino Oligomer (GMO) containing chimeras, which were inaccessible otherwise. Examples of these chimeric backbones include PMO-TMO, GMO-PMO-TMO, PMO-sulfonylphosphoramidate chimera, phosphoramidate-phosphorodiamidate chimera etc.

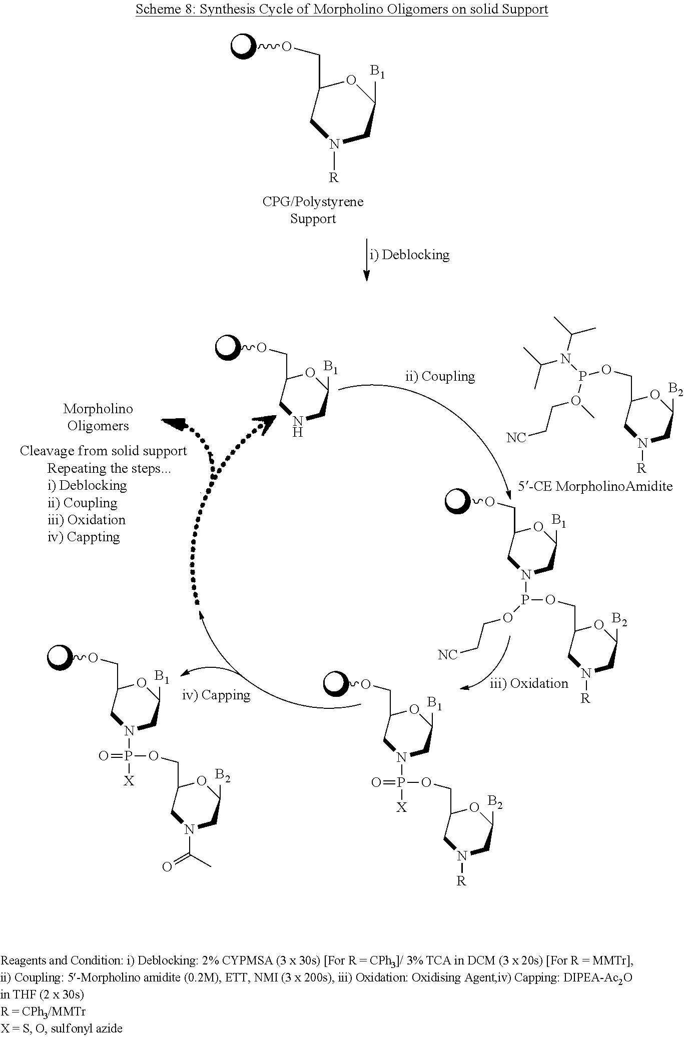

### Oxidising Agents Used:

Thiophosphoramidate: 3-[(Dimethylaminomethylene)amino]-3H-1,2,4-dithiazole-5-thione (DDTT) in pyridine.

Phosphoramidate: (1S)-(+)-(10-camphorsulfonyl)-oxaziridine (CSO) in anhyd. MeCN

Sulfonylphosphorodiamidate: RSO₂N₃ in MeCN or MeCN-THF.

### Convergent Synthesis of PMO Using 5′-Amidite of Short PMO Fragments:

PMO synthesis generally involves a linear synthetic protocol, where a monomer unit (“amidate” or “amidite” monomers) is linked with a growing oligomer chain. However, this approach may lead to lower yields due to inefficient coupling through the formation of N-1 and N-2 mers, one or two subunits shorter than the desired PMO. Separating these shorter mers is challenging with standard purification methods for oligonucleotides, as they share almost identical polarity, ultimately affecting the overall purity of the PMO. Thus, employing a fragment or block ligation approach would be effective. It also reduces the number of coupling steps necessary to achieve the desired PMO. Very recently Wada et. al. attempted convergent synthesis of PMO by H-Phosphonate Chemistry in solution, although the length of PMO was achieved upto only 8-mer (Tsurusaki, T.; Sato, K.; Imai, H.; Hirai, K.; Takahashi, D.; Wada, T. Convergent synthesis of phosphorodiamidate morpholino oligonucleotides (PMOs) by the H-phosphonate approach. Sci. Rep. 2023, 13(1), 12576).

The present method for convergent synthesis involves synthesis of 3-6 mer PMO fragments in solution, keeping free —OH at the 5′ end, which is then activated to 5′-phosphoramidite PMO fragment. This activated fragment is then coupled to 3′-NH of the growing oligomer chain, followed by oxidation (Scheme 9), as discussed above. The method is simple and robust and can be easily applied for the synthesis of a full length PMO either in solid supports or in solution phase.

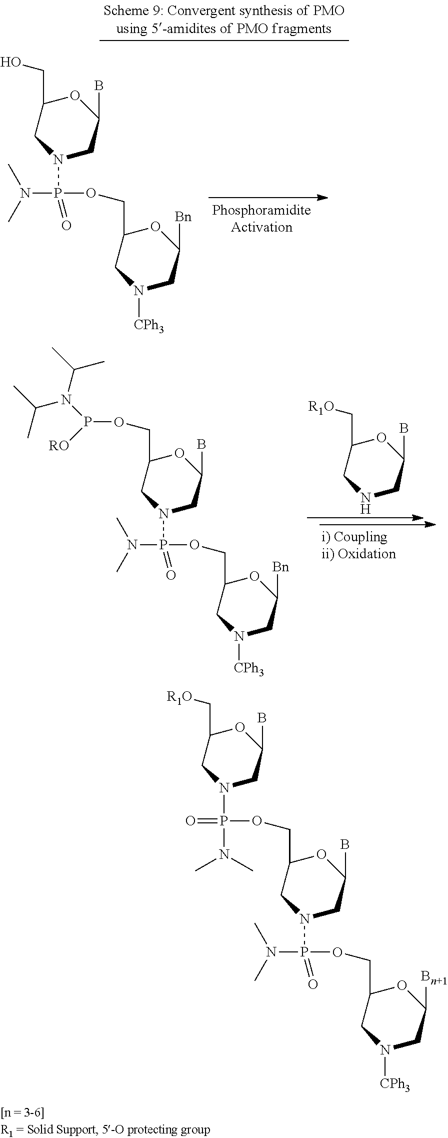

This convergent approach can be applied to the synthesis of diverse chimeric backbone consisting of thiophosphoramidate, phosphorodiamidate, phosphoramidate and sulfonyl-phosphorodiamidate linkages (Scheme-10).

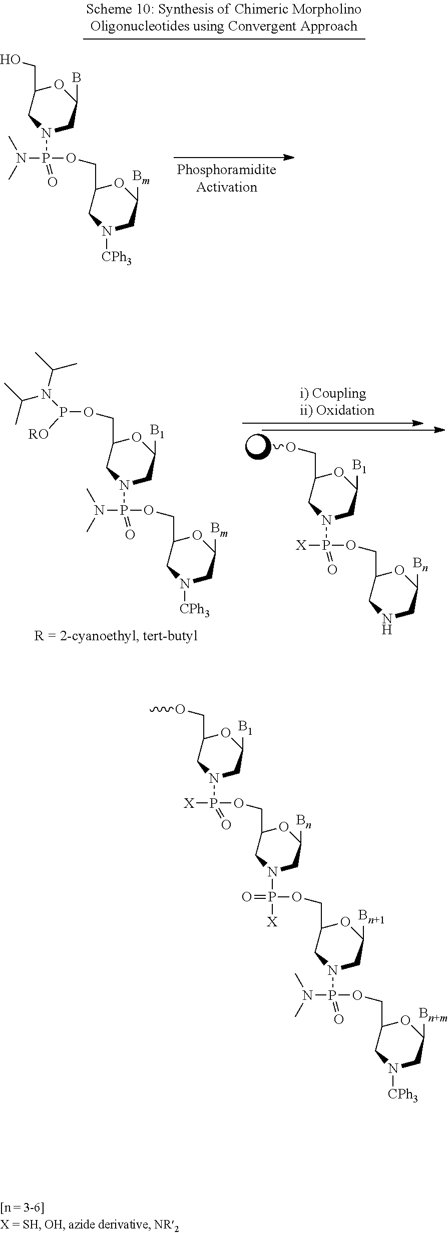

Guanidium linkage is another class of modification in PMO backbone which makes it self-transfecting (Das, Ujjal.; Kundu, J.; Shaw, P.; Bose, C.; Ghosh, A.; Gupta, S.; Sarkar, S.; Bhadra, J.; and Sinha, S. Self-transfecting GMO-PMO antisense chimera targeting Nanog enable gene silencing in vitro and suppresses tumor growth in 4T1 allografts in mouse. Mol. Ther. Nucleic Acids 2023, 32, 203-228. https://doi.org/10.1016/j.omtn.2023.03.011). After the synthesis of GMO, 5′-morpholino amidites were used to extend the remaining oligo by thiophosphoramidate (TMO) and phosphorodiamidate (PMO) backbone. This approach eventually leads to chimeric backbones like GMO-TMO, GMO-PMO-TMO (Scheme-11), which are expected to have interesting biophysical and biological properties. This chimera is useful for developing next generation antisense oligonucleotides therapy for treating different types of genetic diseases or in general RNA targeted therapies for all types of diseases including bacteria and viruses.

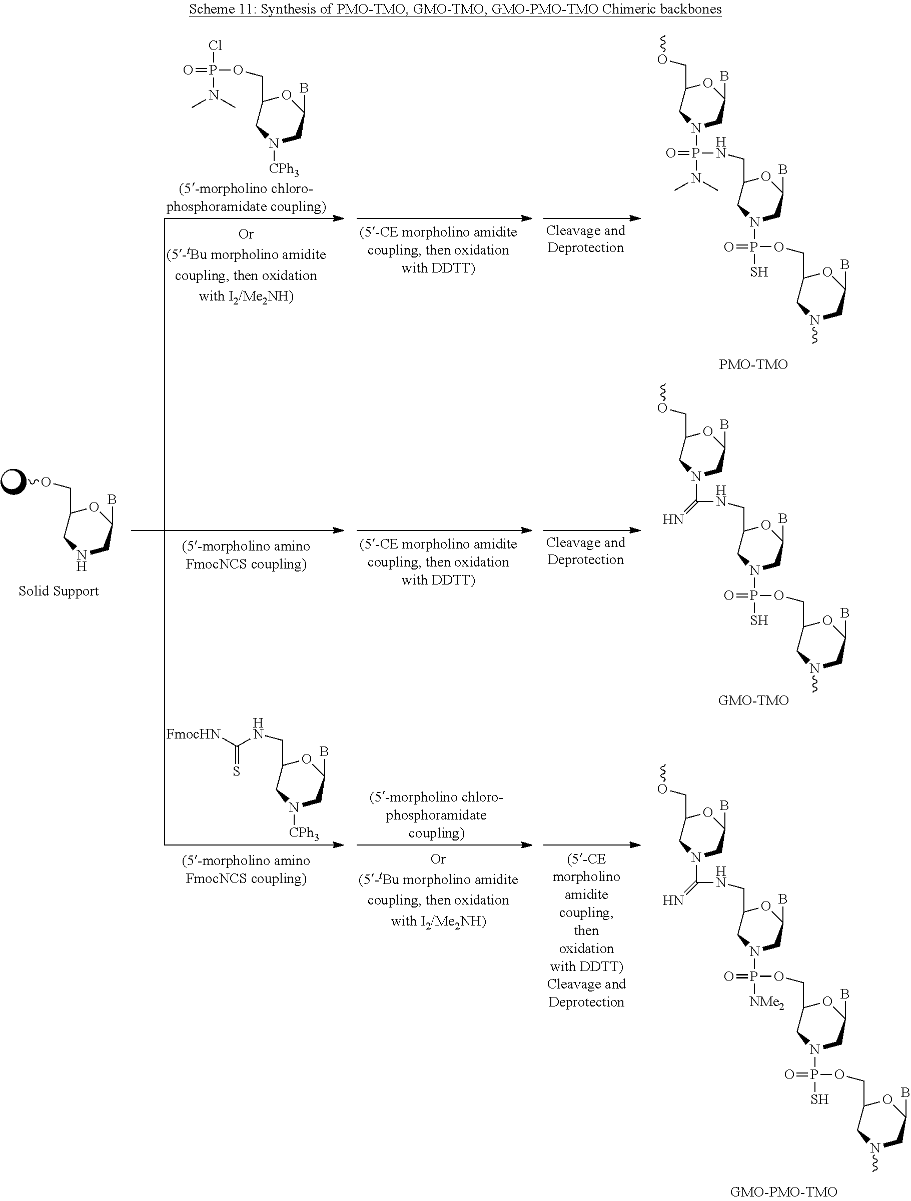

It is noteworthy to mention herein that in chain elongation of these above chimeras, if commercially available 3′-DMTr-protected-5′-phosphoramidites DNA/RNA monomers are used then such chimeras can be extended with DNA/RNA oligos also. Again, a next generation chimeras will be developed.

### General Procedure for the Synthesis of 5′-Morpholino Amidite

The 5′-OH morpholino nucleosides (1A, 1T, 1C, 1G) were dissolved in THF/ACN (8:2) and BTT (l equiv) was added to it. Finally, 2-cyanoethyl-N,N,N′,N′-tetraisopropylphosphorodiamidite reagent was added drop wise to the stirred solution at room temperature. The reaction mixture was left for 40 min and the reaction was quenched by sat. NaHCO₃. Finally, reaction mixture was extracted by ethyl acetate and purified by silica gel column chromatography to afford the 5′-phosphoramidite morpholino nucleosides (2A, 2T, 2G, 2C).

¹H NMR (300 MHz, CDCl₃) δ (ppm) 7.48 (d, J=7.7 Hz, 6H), 7.38-7.24 (m, 6H), 7.19 (d, J=7.2 Hz, 3H), 7.04 (d, J=1.5 Hz, 1H), 6.15 (dd, J=9.6, 2.0 Hz, 1H), 4.38-4.25 (m, 1H), 3.76-3.44 (m, 6H), 3.36 (dt, J=11.3, 2.5 Hz, 1H), 3.21 (dd, J=11.8, 2.2 Hz, 1H), 2.59-2.39 (m, 2H), 1.82 (d, J=1.2 Hz, 3H), 1.43 (d, J=9.6 Hz, 1H), 1.31-1.19 (m, 1H), 1.15 (d, J=6.8 Hz, 6H), 1.06 (dd, J=14.6, 6.8 Hz, 6H).

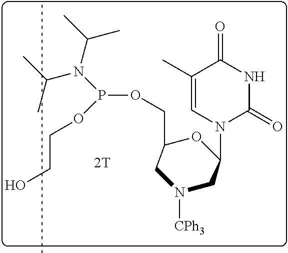

¹³C NMR (75 MHz, CDCl₃) δ (ppm) 163.89, 150.11, 135.73, 129.38, 128.07, 126.66, 117.66, 110.66, 110.62, 80.73, 77.00, 76.36, 64.19, 63.98, 58.62, 58.37, 52.28, 50.04, 43.38, 43.35, 43.21, 43.18, 24.79, 24.76, 24.70, 24.67, 20.58, 20.53, 20.49, 20.44, 12.59.

³¹P NMR (121 MHz, CDCl₃) δ (ppm) 149.38, 149.25.

HRMS (ESI) [M+H]⁺: Calculated mass for C₃₈H₄₇N₅O₅P=684.3315, found 684.3324.

Yield: 86%

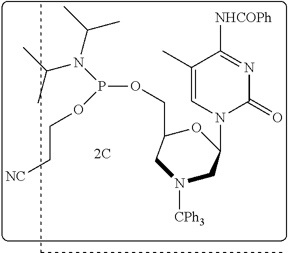

¹H NMR (300 MHz, CDCl₃) δ (ppm) 7.88 (dd, J=7.3, 1.8 Hz, 2H), 7.70 (d, J=7.5 Hz, 1H), 7.60-7.40 (m, 8H), 7.28 (t, J=8.0 Hz, 6H), 7.17 (t, J=7.2 Hz, 3H), 6.25 (dd, J=9.3, 2.3 Hz, 1H), 4.38-4.26 (m, 1H), 3.78-3.42 (m, 7H), 3.22 (d, J=11.7 Hz, 1H), 2.51 (dt, J=12.9, 6.4 Hz, 2H), 1.57-1.45 (m, 1H), 1.26 (d, J=2.7 Hz, 2H), 1.15 (t, J=5.8 Hz, 6H), 1.06 (dd, J=14.6, 6.8 Hz, 6H).

¹³C NMR (75 MHz, CDCl₃) δ (ppm) 163.28, 155.00, 145.55, 134.12, 130.20, 129.93, 128.88, 128.71, 127.46, 118.51, 97.63, 83.05, 77.90, 77.39, 77.29, 65.10, 64.89, 59.44, 59.19, 53.90, 50.89, 46.23, 44.19, 44.03, 25.65, 25.61, 25.55, 25.52, 25.49, 21.37, 21.28.

³¹P NMR (121 MHz, CDCl₃) δ (ppm) 149.41, 149.25.

HRMS (ESI) [M+H]⁺: Calculated mass for C₄₄H₅₀N₆O₅P=773.3580, found 773.3576.

Yield: 80%

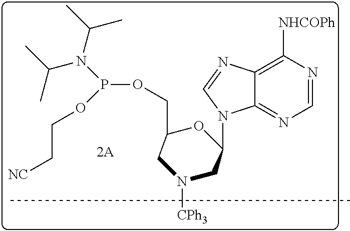

¹H NMR (300 MHz, CDCl₃) δ (ppm) 9.74 (s, 1H), 8.68 (s, 1H), 8.00-7.84 (m, 3H), 7.65-7.08 (m, 18H), 6.39 (dt, J=9.6, 2.0 Hz, 1H), 4.40 (td, J=6.4, 3.2 Hz, 1H), 3.80-3.39 (m, 7H), 3.30 (d, J=11.2 Hz, 1H), 2.55-2.33 (m, 2H), 1.79 (td, J=10.6, 5.6 Hz, 1H), 1.69-1.53 (m, 1H), 1.16-0.95 (m, 12H).

¹³C NMR (75 MHz, CDCl₃) δ (ppm) 165.45, 152.76, 151.54, 149.89, 140.93, 133.88, 132.73, 129.38, 128.79, 128.21, 128.15, 126.78, 123.25, 117.78, 80.38, 77.12, 76.52, 76.42, 76.26, 76.17, 64.22, 64.00, 60.54, 58.67, 58.63, 58.42, 58.38, 53.39, 50.15, 43.38, 43.36, 43.22, 43.19, 24.81, 24.78, 24.72, 20.57, 20.54, 20.48, 20.45, 14.42.

³¹P NMR (121 MHz, CDCl₃) δ (ppm) 149.52, 149.25.

HRMS (ESI) [M+H]⁺: Calculated mass for C₄₅H₄₉N₈O₄P=797.3672, found 797.3692.

Yield: 76%

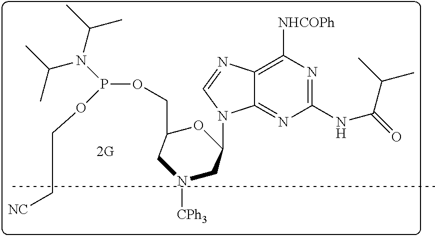

¹H NMR (300 MHz, CDCl₃) δ (ppm) 8.00 (d, J=7.3 Hz, 1H), 7.54 (s, 1H), 7.48-7.33 (m, 6H), 7.23 (t, J=7.5 Hz, 7H), 7.12 (t, J=7.3 Hz, 3H), 6.06-5.94 (m, 1H), 5.87 (d, J=7.8 Hz, 1H), 4.28 (q, J=8.3, 7.2 Hz, 1H), 4.11 (ddd, J=14.4, 7.2, 2.4 Hz, 3H), 3.73-3.32 (m, 10H), 2.70-2.62 (m, 2H), 2.46 (d, J=6.4 Hz, 2H), 1.31-1.15 (m, 24H), 1.15-0.91 (m, 13H).

¹³C NMR (75 MHz, CDCl₃) δ(ppm) 179.37, 156.02, 148.14, 148.08, 148.04, 136.49, 129.34, 128.06, 126.71, 120.99, 117.99, 117.90, 117.21, 80.47, 76.28, 76.02, 75.93, 63.90, 58.57, 58.49, 58.45, 58.42, 58.37, 58.32, 58.22, 53.20, 53.06, 50.18, 50.04, 45.57, 45.55, 45.48, 45.46, 43.31, 43.14, 36.23, 24.76, 24.74, 24.66, 23.16, 23.13, 23.10, 23.07, 20.61, 20.56, 20.53, 20.48, 20.26, 20.16, 19.33, 19.02, 19.00.

³¹P NMR (121 MHz, CDCl₃) δ(ppm) 149.51, 148.77, 14.17.

HRMS (ESI) [M+H]⁺: Calculated mass for C₄₂H₅₁N₈O₅P=779.3828, found 779.3799.

Yield: 72%

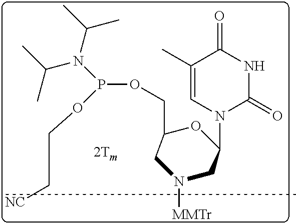

¹H NMR (300 MHz, CDCl₃) δ (ppm) 7.60-7.11 (m, 12H), 7.07 (d, J=1.4 Hz, 1H), 6.94-6.80 (m, 2H), 6.15 (dt, J=9.6, 2.2 Hz, 1H), 4.31 (qd, J=5.1, 2.4 Hz, 1H), 3.80 (s, 3H), 3.79-3.47 (m, 6H), 3.37 (dt, J=11.3, 2.4 Hz, 1H), 3.22 (dt, J=11.9, 2.3 Hz, 1H), 2.62-2.48 (m, 2H), 1.85 (d, J=1.1 Hz, 3H), 1.59-1.36 (m, 2H), 1.21-1.04 (m, 12H).

¹³C NMR (75 MHz, CDCl₃) δ (ppm) 163.84, 158.01, 150.03, 135.68, 130.58, 129.13, 127.97, 126.45, 117.59, 113.27, 110.56, 110.52, 80.66, 77.58, 77.16, 76.74, 76.38, 76.27, 76.09, 76.00, 64.19, 64.09, 63.97, 63.88, 60.48, 58.54, 58.52, 58.30, 58.27, 55.29, 52.15, 52.09, 49.90, 43.27, 43.24, 43.11, 43.08, 24.70, 24.67, 24.60, 24.57, 21.13, 20.48, 20.43, 20.39, 20.34, 14.29, 12.50.

³¹P NMR (121 MHz, CDCl₃) δ (ppm) 149.30, 149.19.

HRMS (ESI) [M+H]⁺: Calculated mass for C₃₉H₄₈N₅O₆P=714.3438, found 714.3421.

Yield: 82%

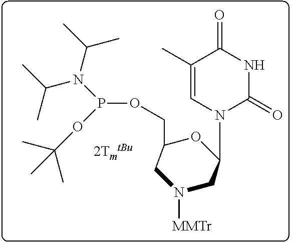

¹H NMR (300 MHz, CDCl₃) δ (ppm) 8.21 (s, 1H), 7.62-7.01 (m, 14H), 6.91-6.75 (m, 2H), 6.08 (dt, J=9.4, 2.0 Hz, 1H), 4.27 (d, J=8.2 Hz, 1H), 3.78 (s, 3H), 3.69-3.39 (m, 5H), 3.39-3.20 (m, 2H), 1.83 (d, J=1.2 Hz, 3H), 1.62 (s, 2H), 1.30 (s, 5H), 1.27-1.17 (m, 9H), 1.17-0.97 (m, 12H).

³¹P NMR (121 MHz, CDCl₃) δ (ppm) 138.89, 138.63, 7.01, 6.20.

HRMS (ESI) [M+H]⁺: Calculated mass for C₄₀H₅₃N₄O₆P=717.3750, found 717.3780.

Yield: 71%

Following the above protocol, 5′-TTT-CPh₃ block was activated to obtain the phosphoramidite.

HRMS (ESI) [M+H]⁺: Calculated mass for C₆₂H₈₄N₁₃O₁₅P₃=1344.5469, found 1344.5500

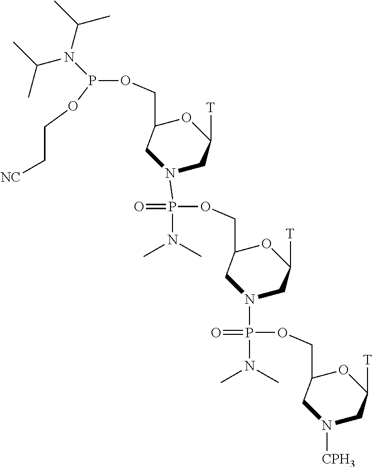

### Solution Phase Synthesis of Dimer and Tetramer Using Phosphoramidite Morpholino Monomer:

First 5′ TBDPS or DMTr protected morpholino 3′-NH monomers with 5′-morpholino amidite or phosphoramidite PMO block were stirred in MeCN solution, then ETT or BTT activator and N-methyl imidazole (NMI) were added. It was left for 10 min and the reaction mixture was oxidised by I₂ and dimethyl amine (2.0M in THF) to produce the desired phosphorodiamidate dimer.

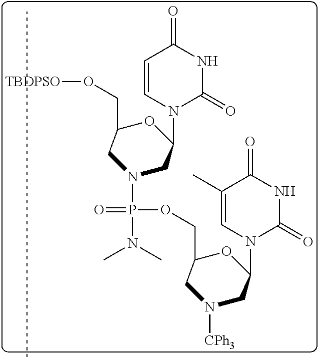

¹H NMR (300 MHz, CDCl₃) δ (ppm) 10.22 (d, J=15.3 Hz, 1H), 10.00 (d, J=19.2 Hz, 1H), 7.66 (dh, J=5.6, 2.6, 1.7 Hz, 4H), 7.58-7.30 (m, 13H), 7.27-7.06 (m, 6H), 6.30-6.15 (m, 1H), 5.71 (t, J=8.8 Hz, 1H), 4.40 (d, J=11.5 Hz, 1H), 4.09-3.64 (m, 5H), 3.55-3.30 (m, 3H), 3.16 (t, J=8.9 Hz, 1H), 2.65 (dd, J=11.7, 8.7 Hz, 6H), 1.88-1.76 (m, 3H), 1.61-1.39 (m, 2H), 1.08 (d, J=4.5 Hz, 8H).

¹³C NMR (75 MHz, CDCl₃) δ (ppm) 163.99, 163.52, 162.63, 150.24, 150.19, 149.87, 149.81, 139.27, 135.55, 135.51, 133.11, 132.98, 132.86, 132.82, 129.95, 129.16, 127.90, 127.80, 126.48, 110.65, 110.61, 102.49, 80.43, 79.91, 79.79, 77.83, 77.58, 77.36, 77.16, 76.82, 76.74, 75.25, 65.39, 64.16, 51.80, 49.16, 47.37, 45.16, 36.72, 36.70, 36.66, 36.64, 36.48, 31.46, 26.79, 19.28, 19.24, 12.44, 12.36.

³¹P NMR (121 MHz, CDCl₃) δ (ppm) 16.51, 16.23.

Yield: 71%

In the case of phosphoramidite PMO block (TTT) was coupled with 5′ TBDPS-U monomer to obtain tetramer PMO 5′-TBDPS-UTTT-CPh₃-3′ by convergent approach (Scheme 9).

HRMS (ESI) [M+Na]⁺: Calculated mass for C₈₀H₁₀₂N₁₅O₁₉P₃SiNa=1720.6356, found 1720.6355.

### Synthesis of PMO, TMO and TMO-PMO Chimera in Automated Synthesizer (H-8, K & A Laborgeraete)

There are 6 different reagents bottle connected with the machine which are
I) Washing
II) CAP A
III) CAP B
IV) Deblocking
V) Activator
VI) Oxidizer.
   Total 12 amidite bottles can be connected at a time. (i) 4 normal position (A, T, G and C) for normal amidites, (ii) 4 modified position (Z, O, S and U) for modified amidites, (iii) 4 additional position (1, 2, 3 and 4) for very small scale synthesis. For the synthesis of PMO normal A, T, G and C positions were accessed.
   Each automated synthesis cycle is composed of five steps:
I) Deblocking
II) Neutralization
III) Coupling
IV) Oxidation
V) Capping

### I) Deblocking:

Step 1—Gas flow for is through the column from the bottom to the waste.
Step 2—1 see ACN was purged with is delay through the column from the bottom to the waste.
Step 3—Repeat of Step 1 and Step 2 for another two times.
Step 4—1.2 s of deblocking cocktail 3% TCA-DCM (TCA: trichloroacetic acid) or CYPMSA (Cyanopyridine-Methanesulfonic Acid salt) was purged with 60 s (for CYPMSA)/20 s (3% TCA in DCM) delay through the column from the bottom to the trityl monitor.
Step 5—Repeat of Step 4 for another 2 times.
Step 6—2 s ACN and gas was purged with is delay through the column from the bottom to the waste.

### II) Neutralization:

Step 1—1 s of neutralizing solution (20% DIPEA-THF) was purged with 30 s delay.
Step 2—Repeat of Step 1 for another 2 times.
Step 3—2 s ACN and gas was purged repeatedly with is delay through the column from the bottom to the waste

### III) Coupling:

Step 1—0.3 sec of 5′-morpholino amidite monomer (0.2 M) and activator of ETT (0.12 M)/NMI (0.7M) were purged by mixing with 4 min delay through the column from the bottom to the waste.
Step 2—Repeat of Step 1 for another 2 times
Step 3—Gas flow for 10 s through the column from the bottom to the waste.
Step 4—2 sec ACN and gas were purged repeatedly with is delay through the column from the bottom to the waste.
Step 5—Repeat of Step 4 for two times.

### IV) Oxidation:

Step-1 1.2 sec of oxidising reagent was purged through column and delay 6 min (for DDTT) to 15 min(for I₂/Me₂NH) depending on the oxidizer.
Step-2 Repeat of Step 1 for another 2 times.
Step-3 2 sec ACN and gas were purged repeatedly with is delay through the column from the bottom to the waste.
Step-4 Repeat of Step 3 for another 3 times.

### V) Capping:

Step 1—1.5 sec of CAP A (20% Ac₂O-THF) and CAP B (20% DIPEA-THF) of were purged by mixing with 60 s delay through the column from the bottom to the waste.
Step 2—Repeat of Step 1 for two times.
Step 3—Gas flow for 3 s through the column from the bottom to the waste.
Step 4—2 sec ACN and gas were purged repeatedly with is delay through the column from the bottom to the waste.
Step 5—Repeat of Step 4 for another 2 times

It is thus possible for the present advancement to provide for monomers and phosphorodiamidate morpholino oligonucleotides (PMO) thereof comprising 5′-phosphoramidite morpholino monomers (2) as Trityl or monomethoxytrityl (MMTr)-protected 5′-phosphoramidite morpholino monomers and their method of synthesis, which monomers not only facilitates linear but also facilitate convergent synthesis of PMO from solution to solid phase support including on CPG (controlled pore glass) or polystyrene resin support. Advantageously, said 5′-phosphoramidite morpholino monomer and block are stable in anhydrous acetonitrile solvent up to 3 days in room temperature.

Synthesis of Thiophosphoramidate Morpholino Oligonucleotides (TMO) in 5′→3′ direction using Phosphoramidite chemistry could be made possible that is also extendable to chiral Thiophosphoramidate Morpholino synthesis. PMO-TMO, PMO-sulfonylphosphoramidate chimera, phosphoramidate-phosphorodiamidate chimera could all be synthesized by using both 5′-CE morpholino amidite monomers and 5′-Bu morpholino amidite monomers, including GMO-PMO-TMO, GMO-TMO chimera by the present phosphoramidite monomers and method thereof. The present monomers and method thereof also enables synthesis of PMO-DNA/PMO-RNA chimera using commercially available 5′-phophoramidite of 3′-DMTr protected DNA and RNA amidites and here also the synthesis rolls in 5′→3′ direction without hampering the Watson-Crick base pairing.

The following oligos were synthesized by automated oligo synthesizer. Oligos were characterized by HPLC and mass analysis as given under the Figures.

i.
PMO 5′-TTTTTTT-MMTr 3′
ii.
PMO 5′-TTTTTTTTTTTTTTTTTTT-MMTr 3′
iii.
TMO 5′-TₛTₛT_{Ss}T-Tr (CPh3)-3′
iv.
PMO-TMO chimera 5′-TTTₛTₛT-CPh₃-3′[S stands-TMO linkages]
v.
GMO-PMO-TMO chimera 5′-TgTTₛT-CPh3-3′:

[g = guanidinium linkage; s = Thiophosphoramidate linkage]

We claim:

1. Monomers and phosphorodiamidate morpholino oligonucleotides (PMO) thereof comprising 5′-phosphoramidite morpholino monomers (2) as Trityl or monomethoxytrityl (MMTr)-protected 5′-phosphoramidite morpholino monomers as represented hereunder:

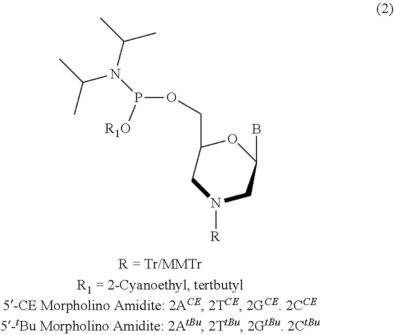

2. The monomers as claimed in claim 1 wherein said nucleobase (B) in said monomers includes all four nucleobases: adenine, thyamine, gunanine, cytosine based monomers for each 2-cyanoethyl (CE) and tert-butyl (^{t}Bu) based phosphoramidites.

3. The monomers as claimed in claim 1 are stable in anhydrous acetonitrile solvent up to 3 days in room temperature.

4. A process for the synthesis of monomers and phosphorodiamidate morpholino oligonucleotides (PMO) thereof as claimed in claim 1 comprising the steps of
(i) Providing 3′-NH, 5′-OH morpholino monomers with nucleobases protected with regular amide-based protecting groups including benzamide for A and C, iso-butyramide for G;
(ii) Protecting the 3′-NH end with Trityl or monomethoxytrityl (MMTr) groups to obtain 3′-N Trityl or monomethoxytrityl (MMTr) protected 5′-OH morpholino monomers;
(iii) Adding 2-cyanoethyl-N,N,N′,N′-tetraisopropylphosphorodiamidite reagent (for 5′-CE Morpholino Amidites) or tert-butyl-N,N,N′,N′-tetraisopropylphosphorodiamidite reagent (for 5′-^{t}Bu Morpholino Amidites) to said 3′-N Trityl or monomethoxytrityl (MMTr) protected 5′-OH morpholino monomers dissolved in THF-ACN (8:2) or THF:DCM (8:2) or DCM in presence of activators to activate N,N-diisopropyl component of said tetraisopropylphosphorodiamidite reagent at room temperature and obtaining therefrom 5′-CE/5′-^{t}Bu based said 5′-phosphoramidite morpholino monomers (2) by quenching the reaction mixture by saturated NaHCO₃ and extracting with solvent including ethyl acetate to further enable dimers and phosphorodiamidate morpholino oligonucleotides (PMO) therefrom.

5. The process for the synthesis of monomers and phosphorodiamidate morpholino oligonucleotides (PMO) thereof as claimed in claim 4 wherein said activators are 5-Ethylthio-1H-tetrazole (ETT) or 5-Benzylthio-1H-tetrazole (BTT) as activator, in combination with N-methyl imidazole (NMI).

6. The process for the synthesis of monomers and phosphorodiamidate morpholino oligonucleotides (PMO) thereof as claimed in claim 4 wherein for preparation of said dimers by chain extension at 3′-NH end of morpholino
said 5′-CE/5′-^{t}Bu phosphoramidite morpholino monomers (2), and, 5′-TBDPS (tert-butyldiphenylsilyl) or DMTr (Dimethoxytrityl) protected morpholino 3′-NH monomers are taken in MeCN solution in presence of said activators ETT or BTT and in combination with said N-methyl imidazole (NMI) as activators of said N,N-diisopropyl component of said 5′-CE/5′-^{t}Bu Morpholino amidite monomers (2) for forming the P—N bond in about ≈10 mins, followed by oxidation of the P(III) centre to P(V) centre by employing I₂ and dimethyl amine (Me₂NH)≈2.0M in THE as oxidizing agent to produce the desired phosphorodiamidate dimer (4) that is Trityl or monomethoxytrityl (MMTr) dimer.

7. The process for the synthesis of monomers and phosphorodiamidate morpholino oligonucleotides (PMO) thereof as claimed in claim 4 wherein said phosphorodiamidate morpholino oligonucleotides (PMO) is synthesized in automated Oligo synthesizer for solid phase synthesis by employing 5′-Morpholino Amidites (2) as Trityl or monomethoxytrityl (MMTr)-protected 5′-phosphoramidite morpholino monomers (2) based on the steps of:
attaching the 5′-OH morpholino monomers to (Controlled pore glass)/polystyrene support having nucleobases protected with regular amide-based protecting groups including benzamide for A and C, iso-butyramide for G to CPG and having morpholino 3′-NH end protected with Trityl or monomethoxytrityl (MMTr) to allow chain extension at said 3′-NH end of morpholino;
deblocking the Trityl protection with 2% CYPMSA (3-Cyanopyridine-Methanesulfonic Acid salt) or monomethoxytrityl (MMTr) protection with 3% TCA (trichloroacetic acid) in DCM;
coupling with 5′-^{t}Bu-phosphoramidite morpholino monomers (2) of about 0.2 M concentration as Trityl or monomethoxytrityl (MMTr)-protected 5′-^{t}Bu-phosphoramidite morpholino monomers in presence of ETT, NMI, followed by oxidation of P(III) to P(V) state in presence of 0.05M I₂/2M Me₂NH in THF to obtain the dimer, followed by, capping N-end of the dimer away from the support by N,N-Diisopropylethylamine/acetic anhydride and continuing the cycle to obtain said phosphorodiamidate morpholino oligonucleotides (PMO) therefrom having —P—NMe₂ linkages at P(V);
cleaving the thus obtained phosphorodiamidate morpholino oligonucleotides (PMO) from the solid support by 30% aqueous NH₃ at 55° C. for 16 h.

8. The process for the synthesis of monomers and phosphorodiamidate morpholino oligonucleotides (PMO) thereof as claimed in claim 4 wherein
said phosphorodiamidate morpholino oligonucleotides (PMO) as Thiophosphoramidate Morpholino Oligonucleotides (TMO) are synthesized by involving 5′-CE morpholino amidites as Trityl or monomethoxytrityl (MMTr)-protected 5′-CE-phosphoramidite morpholino monomers (2) followed by said oxidation of P(III) to P(V) state in presence of 3-[(Dimethylaminomethylene)amino]-3H-1,2,4-dithiazole-5-thione (DDTT) (0.1M) in pyridine, and obtaining therefrom said dimers and Thiophosphoramidate Morpholino Oligonucleotides (TMO) thereof;
said phosphorodiamidate morpholino oligonucleotides (PMO) as Sulfonylphosphoramidate Morpholino Oligonucleotides are synthesized by involving 5′-CE morpholino amidites as Trityl or monomethoxytrityl (MMTr)-protected 5′-phosphoramidite morpholino monomers (2) followed by said oxidation of P(III) to P(V) state in presence of RSO₂N₃/Aryl sulfonyl azide, where R includes Long chain alkyl group, benzyl, methyl, ethyl in MeCN or MeCN-THF, and obtaining therefrom said dimers and Sulfonylphosphoramidate Morpholino Oligonucleotides thereof;
said dimers and incorporation of monomers in continuing cycle of liquid/solid phase synthesis enables attainment of oligonucleotide chimers including Guanidinium linked Morpholino Oligomer (GMO) containing chimeras, PMO-TMO chimers, GMO-PMO-TMO chimers, PMO-sulfonylphosphoramidate chimers, phosphoramidate-phosphorodiamidate chimers.

9. The process for the synthesis of monomers and phosphorodiamidate morpholino oligonucleotides (PMO) thereof as claimed in claim 4 wherein preparation of dimers and morpholino oligonucleotides (PMO) thereof by chain extension at 5′-OH end of morpholino towards convergent synthesis leading to 3-6 mer PMO fragments in solution or in solid phase is based on the steps of:
keeping free —OH at the 5′ end of morpholino and keeping protected -3′ N end of morpholino by Trityl group which said —OH at 5′ end is then activated to provide 5′-phosphoramidite PMO fragment block that is said trityl protected at its -3′ N end; allowing convergent coupling of said 5′-phosphoramidite PMO fragment block, with, Trityl or monomethoxytrityl (MMTr) de-protected morpholino 3′-NH monomer/oligomer chain with free 3′-NH end; followed by oxidation, and repeating the cycle, and obtaining therefrom phosphorodiamidate backbone of 3-6 mer PMO fragments (Block) in solution.

10. The process for the synthesis of monomers and phosphorodiamidate morpholino oligonucleotides (PMO) thereof as claimed in claim 7 wherein said Trityl or monomethoxytrityl (MMTr) de-protected morpholino 3′-NH monomer/oligomer chain with free 3′-NH end is attached to solid support at its other end through 5′-O— for said convergent coupling to attain therefrom diverse chimeric backbone based PMOs including thiophosphoramidate, phosphorodiamidate, phosphoramidate and sulfonyl-phosphorodiamidate linkages also including PMO-DNA/PMO-RNA chimera.

11. The process for the synthesis of monomers and phosphorodiamidate morpholino oligonucleotides (PMO) thereof as claimed in claim 4 wherein MMTr-protecting group based on mild acid treatment including 2-3% TCA (Trichloroacetic acid) in DCM/3% DCA (Dichloroacetic acid) in DCM enabled deprotection to generate free —NH for further coupling.

12. The process for the synthesis of monomers and phosphorodiamidate morpholino oligonucleotides (PMO) thereof as claimed in claim 6 wherein said oxidizing agents for Thiophosphoramidate backbone involves 3-[(Dimethylaminomethylene)amino]-3H-1,2,4-dithiazole-5-thione (DDTT) in pyridine; for Phosphoramidate backbone involves (1S)-(+)-(10-camphorsulfonyl)-oxaziridine (CSO) in anhydrous MeCN and for Sulfonylphosphorodiamidate backbone involves said RSO₂N₃ in MeCN or MeCN-THF.
